(19) United States
(12) Patent Application Publication
(10) Pub. No.: US 2026/0216278 A1
(43) Pub. Date: Jul. 30, 2026

(54) VANCOMYCIN PHARMACEUTICAL COMPOSITION AND KIT THEREOF, AND METHOD FOR TREATING BACTERIAL INFECTION BY USING THE SAME

(71) Applicant: Salus Therapeutics Inc., Taipei City (TW)

(72) Inventors: Yong QIU, San Diego, CA (US); Yu-Cheng Tseng, Taipei City (TW); Chia-Chian Hung, New Taipei City (TW); Yu-Yang Chiang, New Taipei City (TW)

(21) Appl. No.: 19/454,470

(22) Filed: Jan. 21, 2026

Related U.S. Application Data
(60) Provisional application No. 63/749,068, filed on Jan. 24, 2025.

(51) Int. Cl. A61K 38/14; A61K 9/00; A61K 9/1271; A61P 31/04

(52) U.S. Cl. CPC A61K 38/14; A61K 9/0019; A61K 9/1271; A61P 31/04

(57) ABSTRACT

Provided is a vancomycin pharmaceutical composition, comprising a liposomal vancomycin, wherein the liposomal vancomycin comprises vancomycin or a pharmaceutical acceptable salt thereof, and a first liposome comprising sphingomyelin and cholesterol. Also provided is a kit of the vancomycin pharmaceutical composition and method for treating bacterial infection by using the vancomycin pharmaceutical composition or the kit of the vancomycin pharmaceutical composition.

### CROSS-REFERENCE TO RELATED APPLICATION

Pursuant to 35 U.S.C. § 119 (e), this application claims the benefit of the priority to U.S. Provisional Patent Application No. 63/749,068, filed on Jan. 24, 2025. The content of the prior application is incorporated herein by its entirety.

### BACKGROUND OF THE INVENTION

### 1. Field of the Invention

The present invention relates to a vancomycin pharmaceutical composition and a kit of the vancomycin pharmaceutical composition. Also, the present invention relates to a method for treating bacterial infection by using the vancomycin pharmaceutical composition or the kit of the vancomycin pharmaceutical composition.

### 2. Description of the Prior Arts

Vancomycin, produced by Amycolatopsis orientalis, is a hydrophilic glycopeptide antibiotic that has been applied in clinical use for over 60 years as a penicillin alternative to treat penicillinase producing strains of Staphylococcus aureus (S. aureus). Vancomycin is one of the most widely used antibiotics for treating serious Gram-positive infections, especially those involving methicillin-resistant S. aureus (MRSA), methicillin-resistant Staphylococcus epidermidis (MRSE), or amoxicillin-resistant enterococci. The main indications of vancomycin include skin and soft tissue infections, osteomyelitis, bacteremia, and endocarditis where MRSA is deemed a possible cause. In addition, vancomycin is used to treat Gram-positive pneumonia, primarily in context of hospital-acquired infections, and it is also used to treat bacterial meningitis caused by penicillin-resistant Streptococcus pneumonia.

Vancomycin is a large (molecular weight: 1449.2 Daltons), hydrophilic glycopeptide antibiotic, which inhibits bacterial cell wall synthesis by binding to the D-alanine D-alanyl linkage terminus of polymerizing peptidoglycan. The protein binding of vancomycin is 40% to 50%, primarily to immunoglobulin A and albumin. Generally, vancomycin is mainly administered through intravenous infusion with large volume of distribution (0.4 L/kg to 1.0 L/kg of body weight). Due to the hydrophilic nature, vancomycin is mostly eliminated through renal excretion with an elimination half-life of 6 hours (h) to 12 h in patients with normal renal function. It has been reported that about 80% to 90% of vancomycin was recovered unchanged in urine within 24 hours after single-dose administration.

Antimicrobial resistance is an urgent global health and socioeconomic crisis recognized by World Health Organization (WHO). Drug-resistant bacterial infections were responsible for an estimated 1.27 million global deaths in 2019. Among the drug-resistant bacterial infections, MRSA is a leading cause of hospital acquired pneumonia (HAP) and ventilator-associated pneumonia (VAP) at mortality rates of 30% to 50% worldwide.

Although vancomycin has been recommended as first-line empirical therapy for treating MRSA pneumonia, suboptimal therapeutic outcomes are mainly due to inadequate dosing as well as poor penetration into lung tissue and alveolar macrophages resulting from inherent pharmacokinetic/pharmacodynamic (PK/PD) issues. Penetration efficiency of vancomycin into tissues is variable and can be affected by inflammation and disease state. For example, with uninflamed meninges, vancomycin concentrations in cerebral spinal fluid ranging from 0 mg/L to approximately 4 mg/L of cerebral spinal fluid have been reported, whereas concentrations of 6.4 mg/L to 11.1 mg/L of cerebral spinal fluid have been reported in the presence of meninges inflammation. Serum vancomycin concentrations in lung tissue ranging from 5% to 41% have been reported in studies of healthy volunteers and patients. Epithelial lining fluid (ELF) penetration efficiency in critically injured patients is highly variable, with an overall blood: ELF penetration ratio of 6:1. Besides, long recognized as an extracellular pathogen, S. aureus can also behave as an intracellular “parasite” that is able to survive within neutrophils and macrophages. Viable intracellular S. aureus can be carried within mobile alveolar macrophages and migrate to draining lymph nodes. Bacteria can persist for 3 to 4 days within phagosome vacuoles before they escape into the cytoplasm, cause lytic cell death of the macrophage, and invade the bloodstream, leading to bacteriemia. As a result, better PK/PD properties and enhanced drug penetration into lung tissue and alveolar macrophages are desired for vancomycin in order to fight the formidable pathogenic characteristic possessed by S. aureus, thereby achieving optimal MRSA pneumonia therapeutic outcomes.

To effectively treat invasive MRSA infection, pharmacodynamic studies propose that a desired ratio of the area under the vancomycin concentration-time curve for 24 h (AUC₂₄) to minimum inhibitory concentration (MIC) of vancomycin should be 400 or more (AUC₂₄/MIC≥400 mg*h/L). In the treatment of MRSA infections, when the MRSA strains have a MIC≤1 μg/mL, the desired ratio could be reached by maintaining serum vancomycin concentration 15 mg/L to 20 mg/L, while the standard dosage of vancomycin (i.v.) is approximately 1.5 g every 12 h. However, for treating MRSA strains with MIC of 2 μg/mL, a study using Monte Carlo simulation suggests even the most aggressive dosing regimen considered (2 g every 12 h) only yielded a probability of target attainment of 57% while a nephrotoxicity probability is upward to 35%. Clinical reports also show higher doses of vancomycin were believed to contribute to acute kidney injury with increasing possibility of a poor outcome for MRSA-infected intensive care unit (ICU) patients.

S. aureus is a highly successful pathogen, particularly in causing lower respiratory tract infections, due to its ability to secrete a wide array of virulence factors. Among these, bacterial toxins play a critical role by modifying and destroying host defense cells, as well as damaging cellular membranes and structures at epithelial surfaces, connective tissues, and blood vessels. These effects can extend beyond the primary site of infection, impacting distant organs and tissues.

A prominent group of these virulence factors is the pore-forming toxins (PFTs), which account for approximately 25% to 30% of all cytotoxic bacterial proteins. PFTs are the largest category of virulence factors and are produced by both Gram-positive and Gram-negative bacteria. During infection, S. aureus secretes PFTs such as hemolysins and leukocidins. These toxins disrupt host cellular integrity, leading to significant tissue damage. Their activity stimulates the release of cytokines, proteolytic enzymes, and reactive oxygen species by recruited neutrophils, resulting in exacerbated tissue destruction. This destruction liberates essential nutrients, including carbohydrates, amino acids, and iron ions, from host cells, creating a favorable environment for bacterial growth and proliferation.

Henry et al. have demonstrated a groundbreaking approach to combat bacterial infections (Nature Biotechnology 33, pages 81-88, 2015). The study highlights the development of engineered liposomes designed to mimic the lipid composition of host cell membranes, allowing them to sequester and neutralize bacterial exotoxins. These liposomes, comprising high concentrations of cholesterol and sphingomyelin, can effectively bind a range of pore-forming toxins, such as α-hemolysin and pneumolysin, protecting mammalian cells in vitro from cytolytic damage.

In vivo experiments showed that the administration of these liposomes significantly improved survival in mouse models of severe infections, including those caused by S. aureus and Streptococcus pneumoniae. The liposomal treatment reduced tissue damage, bacterial loads, and pro-inflammatory cytokine levels (such as TNF-α), thereby preventing the onset of sepsis and cytokine storms. Importantly, the liposomes provided protection without directly killing bacteria, reducing the risk of fostering antibiotic resistance.

A vancomycin composition having a better efficacy is still needed to resolve the increasing drug-resistant bacteria problem. In addition, a prolonged storage period of the composition is also desired.

### SUMMARY OF THE INVENTION

To overcome the shortcomings, the present invention provides a vancomycin pharmaceutical composition, a kit of the vancomycin pharmaceutical composition, and a method for treating bacterial infection by using the vancomycin pharmaceutical composition or the kit of the vancomycin pharmaceutical composition. The vancomycin pharmaceutical composition which can be prepared by the kit of vancomycin pharmaceutical composition can prolong the plasma circulation time of vancomycin and effectively kill multiple drug-resistant organisms, and can be applied to the treatment of infections of multiple drug-resistant organisms, especially infections of methicillin-resistant S. aureus (MRSA), methicillin-resistant Staphylococcus epidermidis (MRSE), or amoxicillin-resistant Enterococcus spp.

In one aspect, the present invention provides a vancomycin pharmaceutical composition, comprising a liposomal vancomycin, wherein the liposomal vancomycin comprises a therapeutically effective amount of vancomycin or a pharmaceutical acceptable salt thereof, and a first liposome comprising sphingomyelin and cholesterol.

In another aspect, the present invention provides a kit of a vancomycin pharmaceutical composition, comprising: (a) a liposomal vancomycin, comprising a therapeutically effective amount of vancomycin or a pharmaceutical acceptable salt thereof, and a first liposome comprising sphingomyelin and cholesterol; and (b) a second liposome, comprising sphingomyelin; wherein (a) the liposomal vancomycin and (b) the second liposome are separately stored.

In another aspect, the present invention provides a method for treating bacterial infection, comprising: administering the above-mentioned vancomycin pharmaceutical composition to a subject in need thereof.

In another aspect, the present invention provides a method for treating bacterial infection, comprising: providing the above-mentioned kit; reconstituting the liposomal vancomycin with the second liposome to obtain a vancomycin pharmaceutical composition; and administering the vancomycin pharmaceutical composition to a subject in need thereof.

In yet another aspect, the present invention provides a method for treating bacterial infection, comprising: providing the above-mentioned kit; reconstituting the liposomal vancomycin with a reconstitution liquid selected from water and an intravenous fluid to obtain a reconstituted liposomal vancomycin; and administering the reconstituted liposomal vancomycin and the second liposome to a subject in need thereof separately or simultaneously.

In the present invention, vancomycin or a pharmaceutical acceptable salt thereof is used as an active pharmaceutical ingredient (API).

In some embodiments, in the liposomal vancomycin, the weight ratio of the vancomycin to the first liposome is from about 9:1 to about 0.06:1, or 8:1 to about 0.1:1, or about 7:1 to about 0.5:1, or about 6:1 to about 0.5:1, or about 5.5:1 to about 1:1, or about 5:1 to about 1:1, or about 5:1 to about 1.5:1, or about 4:1 to about 1.5:1, or about 3.5:1 to about 1.5:1, or about 3.2:1 to about 1.5:1, or about 3.2:1 to about 1.6:1. In the present invention, the weight ratio of the vancomycin to the first liposome is calculated in accordance with the weight of vancomycin.

In some embodiments, the first liposome comprises 40 wt % to 60 wt % of cholesterol and 60 wt % to 40 wt % of sphingomyelin. In some embodiments, the first liposome comprises 40 wt % to 55 wt % of cholesterol and 60 wt % to 45 wt % of sphingomyelin. In some embodiments, the first liposome comprises 40 wt % to 50 wt % of cholesterol and 60 wt % to 50 wt % of sphingomyelin. In some embodiments, the first liposome comprises 45 wt % to 60 wt % of cholesterol and 55 wt % to 40 wt % of sphingomyelin. In some embodiments, the first liposome comprises 50 wt % to 60 wt % of cholesterol and 50 wt % to 40 wt % of sphingomyelin. In some embodiments, the first liposome comprises 50 wt % of cholesterol and 50 wt % of sphingomyelin.

In some embodiments, the vancomycin pharmaceutical composition further comprises a pharmaceutically acceptable excipient. In some embodiments, the first liposome further comprises a pharmaceutically acceptable excipient.

In some embodiments, the pharmaceutically acceptable excipient May be a lyoprotectant, a pharmaceutical acceptable buffering agent, an anti-oxidant, a stabilizer, or a combination thereof.

In some embodiments, the lyoprotectant may be sucrose, glucose, trehalose, lactose, mannitol, sorbitol, inulin, polyvinylpyrrolidone, polyvinyl alcohol, dextran, glycine, arginine, proline, or a combination thereof.

In some embodiments, the pharmaceutical acceptable buffering agent may be a citrate buffering agent (comprising citric acid and sodium citrate), an acetate buffering agent (comprising acetic acid and sodium acetate), a phosphate buffering agent (comprising monobasic sodium phosphate and dibasic sodium phosphate), a histidine buffering agent (comprising histidine and histidine HCl), a MES buffering agent (comprising 2-(N-morpholino) ethanesulfonic acid), a succinate buffering agent (comprising succinic acid and sodium succinate), a tartrate buffering agent (comprising tartaric acid), a maleate buffering agent (comprising maleic acid and sodium maleate), an imidazole buffering agent (comprising imidazole), or a combination thereof.

In some embodiments, the anti-oxidant may be ascorbic acid, sodium ascorbate, sodium metabisulfite, cysteine, glutathione, thiourea, ethylenediaminetetraacetic acid (EDTA), disodium EDTA, citric acid, tartaric acid, phosphoric acid, or a combination thereof.

In some embodiments, the stabilizer is vancomycin stabilizer. In some embodiments, the vancomycin stabilizer may be N-acetyl-D-alanine, but is not limited thereto.

In some embodiments, the vancomycin pharmaceutical composition further comprises a second liposome, wherein the second liposome comprises sphingomyelin.

In some embodiments, the second liposome comprises 100% sphingomyelin. In some embodiments, the second liposome further comprises a lipid selected from cholesterol, phospholipids, phospholipid derivatives, or a combination thereof. In some embodiments, the second liposome comprises 90 wt % to less than 100 wt % of sphingomyelin and greater than 0 wt % to 10 wt % of a lipid selected from cholesterol, phospholipids, phospholipid derivatives, or a combination thereof. In some embodiments, the second liposome comprises a combination of sphingomyelin and cholesterol. In some embodiments, the second liposome comprises 95 wt % to less than 100 wt % of sphingomyelin and greater than 0 wt % to 5 wt % of a lipid selected from cholesterol, phospholipids, phospholipid derivatives, or a combination thereof. In some embodiments, the second liposome comprises 97 wt % to less than 100 wt % of sphingomyelin and greater than 0 wt % to 3 wt % of a lipid selected from cholesterol, phospholipids, phospholipid derivatives, or a combination thereof. In some embodiments, the second liposome comprises 90 wt % to less than 100 wt % of sphingomyelin, greater than 0 wt % to 10 wt % of a lipid selected from cholesterol, and greater than 0 wt % to 10 wt % of a lipid selected from phospholipids, phospholipid derivatives, or a combination thereof.

In the present invention, the phospholipid may be selected from natural phospholipids, synthetic phospholipids, and any combinations thereof. In some embodiments, the natural phospholipid may be selected from phosphatidylcholine, phosphatidylethanolamine, phosphatidylserine, phosphatidylinositol, phosphatidic acid, cardiolipin, and a combination thereof. In some embodiments, the synthetic phospholipid may be selected from 1,2-dimyristoyl-sn-glycero-3-phosphocholine, 1,2-dipalmitoyl-sn-glycero-3-phosphocholine, 1,2-dioleoyl-sn-glycero-3-phosphocholine, 1,2-distearoyl-sn-glycero-3-phosphocholine, 1,2-dimyristoyl-sn-glycero-3-phosphoethanolamine, 1,2-dioleoyl-sn-glycero-3-phosphoethanolamine, and a combination thereof.

In the present invention, the phospholipid derivative may be selected from polyethylene glycol (PEG)-modified phospholipids, lipid-like phospholipid derivatives, cholesterol-containing derivatives, and any combinations thereof. In some embodiments, the PEG-modified phospholipid may be selected from PEG-phosphatidylethanolamine, PEG-phosphatidylcholine, PEG derivatives of a neutral phospholipid, and monomethoxy PEG-(1,2-distearoyl-sn-glycero-3-phosphoethanolamine) (monomethoxy PEG-DSPE), and any combinations thereof. In some embodiments, the neutral phospholipid may be phosphatidylcholine, phosphatidylethanolamine, lysophosphatidylcholine, lysophosphatidylethanolamine, dipalmitoylphosphatidylcholine, dimyristoylphosphatidylcholine, 1,2-dioleoyl-sn-glycero-3-phosphocholin, and any combinations thereof. In some embodiments, the lipid-like phospholipid derivative may be selected from lyso-phospholipids, ether-linked phospholipids, and a combination thereof. In some embodiments, the cholesterol-containing derivative may be selected from PEG-cholesterol, cholesterol derivatives (e.g., cholesteryl hemisuccinate), and a combination thereof.

In the present invention, the liposomal vancomycin comprised in the kit of the vancomycin pharmaceutical composition is in a lyophilized solid form. In the present invention, the liposomal vancomycin comprised in the vancomycin pharmaceutical composition is in a liquid suspension form.

In some embodiments, before administration, the liposomal vancomycin in a lyophilized solid form is reconstituted with the second liposome in a liquid suspension form or a reconstitution liquid, to obtain the liposomal vancomycin in a liquid suspension form. In some embodiments, the reconstitution liquid is selected from water and an intravenous fluid. In the present invention, when the second liposome in a liquid suspension form is used to reconstitute the liposomal vancomycin in a lyophilized solid form, the second liposome comprised in the kit is in a liquid suspension form; when the reconstitution liquid is used to reconstitute the liposomal vancomycin in a lyophilized solid form, the second liposome in the kit may be in a lyophilized solid form or in a liquid suspension form.

In some embodiments, the liposomal vancomycin is in a liquid suspension form. In some embodiments, the liposomal vancomycin in a liquid suspension form has a Z-average size of 60 nanometers (nm) to 180 nm, or 70 nm to 170 nm, or 80 nm to 160 nm, or 90 nm to 150 nm, or 100 nm to 140 nm.

In some embodiments, the vancomycin pharmaceutical composition has an encapsulating efficiency of 3% to 25%. In some embodiments, the vancomycin pharmaceutical composition has an encapsulating efficiency of 5% to 20%. In the present invention, the encapsulating efficiency of the vancomycin pharmaceutical composition is determined by HPLC-UV method. In the present invention, the encapsulating efficiency of the vancomycin pharmaceutical composition is determined by HPLC-UV method using a 100× diluted vancomycin pharmaceutical composition. In the present invention, the 100× diluted vancomycin pharmaceutical composition is filtered by a filter with 100 KDa molecular weight cut off (MWCO) and washed twice by water before encapsulating efficiency determination.

In some embodiments, the vancomycin pharmaceutical composition has a pH value of 3.0 to 7.0, or 4.0 to 7.0, or 4.5 to 7.0, or 4.5 to 6.5.

In some embodiments, the liposomal vancomycin has an encapsulating efficiency of 3% to 25% after reconstitution with the second liposome in a liquid suspension form or a reconstitution liquid selected from water and an intravenous fluid. In some embodiments, the liposomal vancomycin has an encapsulating efficiency of 5% to 20% after reconstitution with the second liposome in a liquid suspension form or a reconstitution liquid selected from water and an intravenous fluid.

In some embodiments, the water is deionized water.

In some embodiments, the intravenous fluid may be normal saline (0.9% NaCl aqueous solution, or abbreviated as S9W), 5% dextrose aqueous solution (D5W), or any other solution suitable for IV injection. In the present invention, the term “intravenous fluid” indicates a fluid that can be injected into veins for dehydration treatment or prevent dehydration.

In some embodiments, the second liposome comprised in the kit of the vancomycin pharmaceutical composition is in a lyophilized solid form or a liquid suspension form. In some embodiments, the second liposome comprised in the vancomycin pharmaceutical composition is in a liquid suspension form.

In some embodiments, the second liposome in a liquid suspension form may be used as a reconstitution liquid for the liposomal vancomycin.

The solutions which can be used for reconstitution known in the art can be used as the reconstitution solution in the present invention. In some embodiments, before administration, the second liposome in a lyophilized solid form is reconstituted with a reconstitution liquid selected from water and an intravenous fluid, to obtain a second liposome in a liquid suspension form. In some embodiments, the intravenous fluid may be normal saline, 5% dextrose aqueous solution (D5W), or any other solution suitable for IV injection.

In some embodiments, the second liposome is in a liquid suspension form. In some embodiments, the second liposome in a liquid suspension form has a Z-average size of 40 nm to 100 nm, or 45 nm to 90 nm, or 50 nm to 80 nm.

In some embodiments, the liposomal vancomycin in a liquid suspension form and the second liposome in a liquid suspension form are administered separately or administered as a mixture.

In some embodiments, the liposomal vancomycin and the second liposome are separately stored in two containers. In some embodiments, the liposomal vancomycin and the second liposome are separately stored in two chambers in one container. In some embodiments, the container may be a bottle, a vial, an ampoule, or a syringe.

In some embodiments, the above-mentioned kit of vancomycin pharmaceutical composition comprises (a) first container comprising a liposomal vancomycin in a lyophilized solid form, and the (b) second container comprising a second liposome in a liquid suspension form for reconstituting the liposomal vancomycin in a lyophilized solid form to obtain a liposomal vancomycin in a liquid suspension form. After the reconstitution, the vancomycin pharmaceutical composition is ready for administration. Before administration, the vancomycin pharmaceutical composition may be further diluted.

In some embodiments, the above-mentioned kit of vancomycin pharmaceutical composition further comprises (c) water or an intravenous fluid, wherein (a) the liposomal vancomycin, (b) the second liposome and (c) water or an intravenous fluid are separately stored. In some embodiments, the above-mentioned kit of the vancomycin pharmaceutical composition may comprise one or more portions of (c) water or an intravenous fluid, and each portion of (c) water or an intravenous fluid is separately stored. In some embodiments, the liposomal vancomycin, the second liposome, and one portion of (c) water or an intravenous fluid are separately stored in three containers. In some embodiments, the liposomal vancomycin, the second liposome, and two portions of (c) water or an intravenous fluid are separately stored in four containers. In some embodiments, the liposomal vancomycin, the second liposome, and one portion of (c) water or an intravenous fluid are separately stored in three chambers in one container. In some embodiments, the liposomal vancomycin, the second liposome, and two portions of (c) water or an intravenous fluid are separately stored in four chambers in one container. In some embodiments, the container may be a bottle, a vial, an ampoule, or a syringe.

In some embodiments, the above-mentioned kit of vancomycin pharmaceutical composition comprises (a) a liposomal vancomycin in a lyophilized solid form, (b) a second liposome in a liquid suspension form, and one portion of (c) water or an intravenous fluid for reconstituting the liposomal vancomycin in a lyophilized solid form to obtain a liposomal vancomycin in a liquid suspension form. After the reconstitution, the liposomal vancomycin in a liquid suspension form and the second liposome in a liquid suspension form can be administered separately; or, the liposomal vancomycin in a liquid suspension form and the second liposome in a liquid suspension form can be mixed to obtain a vancomycin pharmaceutical composition for administration. Before administration, the vancomycin pharmaceutical composition may be further diluted.

In some embodiments, the above-mentioned kit of vancomycin pharmaceutical composition comprises (a) a liposomal vancomycin in a lyophilized solid form, (b) a second liposome in a lyophilized solid form, and one portion of (c) water or an intravenous fluid for reconstituting the second liposome in a lyophilized solid form to obtain a second liposome in a liquid suspension form; and the liposomal vancomycin in a lyophilized solid form can be reconstituted with the second liposome in a liquid suspension form to obtain a vancomycin pharmaceutical composition. After the double reconstitution, the vancomycin pharmaceutical composition is ready for administration. Before administration, the vancomycin pharmaceutical composition may be further diluted.

In some embodiments, the above-mentioned kit of vancomycin pharmaceutical composition comprises (a) a liposomal vancomycin in a lyophilized solid form, (b) a second liposome in a lyophilized solid form, and two portions of (c) water or an intravenous fluid; wherein the two portions of (c) water or an intravenous fluid are used for respectively reconstituting the liposomal vancomycin in a lyophilized solid form and the second liposome in a lyophilized solid form to obtain a liposomal vancomycin in a liquid suspension form and a second liposome in a liquid suspension form; and the liposomal vancomycin in a liquid suspension form and the second liposome in a liquid suspension form can be administered separately; or, the liposomal vancomycin in a liquid suspension form and the second liposome in a liquid suspension form can be mixed to obtain a vancomycin pharmaceutical composition for administration. Before administration, the vancomycin pharmaceutical composition may be further diluted.

In some embodiments, the kit further comprises an instruction for how to use the kit components.

In some embodiments, the vancomycin pharmaceutical composition may be administered through injection or inhalation.

Other objectives, advantages and novel features of the invention will become more apparent from the following detailed description when taken in conjunction with the accompanying drawings.

### BRIEF DESCRIPTION OF THE DRAWINGS

FIG. 1A shows the survival rates of A1 to A5 groups in Animal Test 1.

FIG. 1B shows the survival rates of B1 to B3 groups in Animal Test 1.

FIG. 1C shows the survival rates of C₁ to C₃ groups in Animal Test 1.

FIG. 1D shows the survival rates of D1 to D4 groups in Animal Test 1.

FIG. 2 shows the vancomycin plasma concentrations of E1 and E2 groups of Animal Test 2.

FIG. 3 shows the ΔLog₁₀ (CFU/thigh) of F1 to F6 groups in Animal Test 3.

FIG. 4 shows the ΔLog₁₀ (CFU/lung) of G1 to G10 groups in Animal Test 4.

FIG. 5 shows the vancomycin lung tissue distribution of G5 to G10 groups of Animal Test 4.

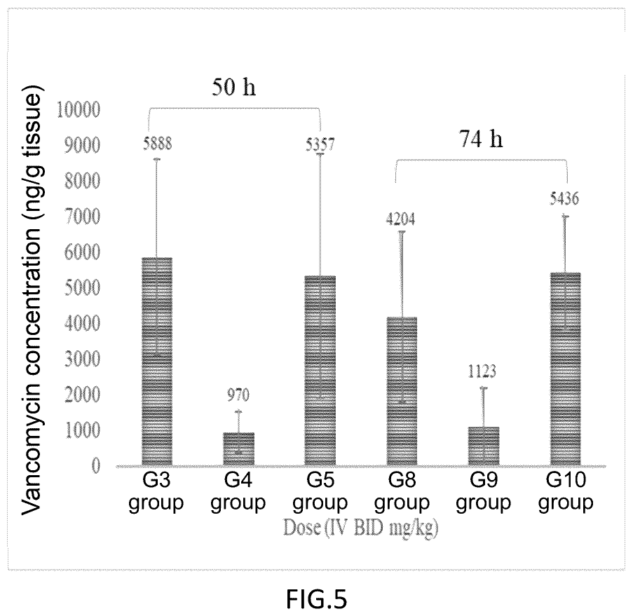

FIG. 6 shows vancomycin plasma concentrations of E1 and E2 groups of Animal Test 5.

### DETAILED DESCRIPTION OF THE PREFERRED EMBODIMENTS

Hereinafter, the present invention will be further illustrated with reference to the following examples. However, these examples are only provided for illustrative purposes, but not to limit the scope of the present invention.

### Preparation Example 1: Preparation of Liposomal Vancomycin (Method I)

Firstly, an “API solution” was prepared. An appropriate amount of vancomycin hydrochloride (Van HCl) was weighted and dissolved in a stirred warm (55° C.) histidine-sucrose buffer solution (containing 20 mM to 40 mM histidine base as the buffering agent and 9% sucrose as the lyoprotectant) in a glass container with a suitable stir bar to obtain the API solution with a flexible Van concentration ranging from 0.66 milligrams per milliliter (mg/mL) to 60 mg/mL, and a controlled pH value of around pH 6.0 to pH 6.5, preferably pH 6.0 to pH 6.2. The Van concentration hereinafter was calculated in accordance with the concentration of the active pharmaceutical ingredient (vancomycin), not calculated in accordance with the salt (Van HCl). The Van concentration prepared in the following Examples could be higher than 60 mg/mL or lower than 0.66 mg/mL as desired as long as the Van remains soluble.

Secondly, a “lipid phase solution of first liposome” was prepared. Cholesterol (Chol) and sphingomyelin (Sm) were fully dissolved in chloroform (CHCl₃) at ambient temperature. The combined lipid concentration of Sm and Chol in chloroform could be flexible from around 100 mg/mL to 300 mg/mL in the chloroform in order to make the total lipid concentration of the liposomal vancomycin flexible from 10 mg/mL to 30 mg/mL.

It should be noted that the acceptable concentrations for Van HCl in the “API solution” and Sm and Chol in the “lipid phase solution of first liposome” are relatively wide. The concentrations provided in this test cover the concentrations tested to be feasible but not their limits.

Next, the lipid phase solution of first liposome was added into the API solution, and the mixture was stirred vigorously without splashing to form a chloroform/water emulsion. Chloroform (b.p. 61.2° C.) was evaporated from the emulsion by controlling the temperature at 55° C. to 58° C. Gradually, a phase-transition from lipid in chloroform/water emulsion to a crude liposomal vancomycin suspension occurred.

The crude liposomal vancomycin suspension could be optionally homogenized by high-shear mixing or sonication to improve homogeneity. With or without homogenization, the residual chloroform of the crude liposomal vancomycin suspension was removed to obtain a liposomal vancomycin suspension. Optionally, the crude liposomal vancomycin suspension was placed under vacuum at room temperature (RT) and a pressure of −68 centimeters mercury column (cm Hg) for 3 hours for further residual chloroform removal.

After removal of chloroform, the crude liposomal vancomycin suspension was subjected to repeated extrusions through a series of polycarbonate track-etched membranes at 50° C. to 60° C. and an elevated pressure to obtain a liposomal vancomycin suspension. After the extrusion, the Z-average size of the liposomal vancomycin suspension should be around 60 nm to 180 nm.

The liposomal vancomycin suspension could be used directly or lyophilized for long term storage. The lyophilization was carried out by a lyophilization process using VirTis Genesis Pilot Freeze Dryer with the following steps:
Freezing temperature and duration: −50° C. for 2 hours;
Ramping: 30 minutes (min) ramp up to −30° C.;
Primary drying 1: −30° C. under 100 millitorr (mT) for 6 hours;
Ramping: 30 min ramp up to −20° C.;
Primary drying 2: −20° C. for 4 hours under 100 mT;
Ramping: 120 min ramp up to 25° C.;
Secondary drying: 25° C. for 2 hours under 100 mT; and
Storage vacuum: 500 mT.

Adjustments and optimizations could be made according to several experimental factors such as different lyophilization machines, different sample loads, different filling volumes, different lyophilization containers, etc.

After the lyophilization process, the lyophilized liposomal vancomycin (i.e., liposomal vancomycin in a lyophilized solid form) was obtained. The lyophilized liposomal vancomycin could be reconstituted by adding back the exact amount of the water that had been removed during the lyophilization process to obtain a liposomal vancomycin suspension (i.e., liposomal vancomycin in a liquid suspension form) before the following examination.

### Preparation Example 2: Preparation of Liposomal Vancomycin (Method II)

The API solution and lipid phase solution of the first liposome were prepared according to methods as described in Preparation Example 1. The API solution and lipid phase solution of the first liposome (approximately 40 mL in total) were introduced in a 1L drying round flask; the round flask was installed onto the rotary evaporator (BUCHI R300 or equivalent, with rotation speed of 150 rpm, hot water bath temperature of 50° C., and chiller temperature of 10° C.). The drying flask containing the API solution and lipid phase solution of the first liposome was fully immersed in the hot water bath and rotated for 3 minutes. Within 20 minutes, the pressure was reduced from 1000 mbar to 350 mbar and maintained at 350 mbar for 5 minutes. Afterward, the pressure was reduced to 150 mbar within 1 minute and maintained at 150 mbar for 10 minutes. The evaporated water was replenished to the original theoretical weight, and the pressure was again reduced to 150 mbar and maintained at 150 mbar for additional 30 minutes. After that, the evaporated water was replenished again to the original theoretical weight. Chloroform was removed by the above process to obtain a crude liposomal vancomycin suspension.

Adjustments and optimizations could be made according to several experimental factors such as rotation speed, temperature, vacuum level, etc. The resulting crude liposomal vancomycin suspension was then subjected to repeated extrusions to obtain a liposomal vancomycin suspension, and the liposomal vancomycin suspension was optionally subjected to lyophilization process as described in Preparation Example 1.

### Test Example 1: Properties of Liposomal Vancomycin

### (1) Particle Size Distribution Analysis of Liposomal Vancomycin

Particle size distribution (PSD) analysis provides several critical qualities of liposomes, such as polydispersity index (PDI) and Z-Average (hydrodynamic size of the ensemble collection of particles measured by dynamic light scattering). For particles size measurement, a dynamic light scattering (DLS) system Zetasizer Nano ZS (Malvern Panalytical) was used following below:
Instrument: Zetasizer Nano ZS (Malvern Panalytical)
Measurement Type: Size
Material: Polystyrene latex (refractive index (RI)=1.59, absorption=0.01)
Dispersant: Water (temperature: 25° C., viscosity: 0.8872 centipoises (cP), RI: 1.33)
General Options: Use dispersant viscosity as sample viscosity
Temperature: 25° C.
Equilibration Time: 120 Seconds
Cell: Disposable cuvettes DTS0012
Angle of Detection: 173° Backscatter (NIBS default)
Measurement Duration: Automatic
Number of Measurements: 3
Delay Between Measurements: 0 Second
Measurement Position: 4.65 mm
Attenuator: Automatic
Analysis Model: General Purpose

The liposomal vancomycin suspension (either before lyophilization or after water-reconstitution) was 20× diluted with deionized water and mixed well. The diluted liposomal vancomycin suspension had a total lipid concentration of approximately 0.5 mg/mL, which was added into a disposable cuvette DTS0012 for measurement. At last, the Z-average size (diameter in nm) and PDI of the liposomal vancomycin suspension was obtained and reported.

### (2) Total Vancomycin Assay of Liposomal Vancomycin

The total vancomycin concentration of the liposomal vancomycin suspension (either before lyophilization or after water-reconstitution) was determined by a HPLC-UV method. The settings of HPLC-UV method are listed below:
Column: Waters XBridge C18, 3.5-μm, 4.6×150-mm or equivalent
Mobile Phase: Gradient of Mobile Phase A and Mobile Phase B, with a gradient program as described in Table 1
Mobile Phase A: ACN/MeOH/solution A=Mar. 4, 1993 (solution A: 7 g/L Tris Buffer, pH 8.05)
Mobile Phase B: ACN/MeOH/solution A=10/40/50
Detector: UV at 280-nm
Temperature: 35° C. for column, 4° C. for sample
Flow Rate: 1.0 mL/min
Injection Volume: 20 μL
Run Time: 65 minutes

TABLE 1

| Time | Flow Rate | Mobile Phase A | Mobile Phase B |
|---|---|---|---|
| (min) | (mL/min) | (%) | (%) |
| 0 | 1.0 | 92 | 8 |
| 27 | 1.0 | 92 | 8 |
| 43 | 1.0 | 80 | 20 |
| 55 | 1.0 | 55 | 45 |
| 56 | 1.0 | 35 | 65 |
| 60 | 1.0 | 35 | 65 |
| 61 | 1.0 | 92 | 8 |
| 65 | 1.0 | 92 | 8 |

In a 50 mL volumetric flask, 500 mg of the liposomal vancomycin suspension (either before lyophilization or after water-reconstitution) and 25 mL of methanol were added and mixed well by gently wobbling to obtain a clear and transparent solution. The solution was sonicated for 10 minutes and cooled to room temperature. Deionized water was added to volume (50 mL in total) and mixed well by stirring for about 10 minutes to obtain a sample solution. The sample solution was centrifuged at 13,000 rpm for 10 minutes, then the upper clear solution was taken for total vancomycin assay per HPLC settings above against an external standard of Van HCl. The vancomycin assay would determine the total vancomycin concentration (Cₜₒₜₐₗ) of the liposomal vancomycin suspension.

### (3) Encapsulating Efficiency Determination of Liposomal Vancomycin Suspension

Encapsulating efficiency (EE) is considered one of the critical qualities of API encapsulated liposome formulations. To determine encapsulating efficiency, separation of the free form vancomycin and liposomal vancomycin (liposome-encapsulated vancomycin) was carried out by ultracentrifugation method. In this invention, a well-known ultracentrifugation method (Method EE-A) was used, but it was found that Method EE-A was acceptable for low-concentration formulations (such as, Van 0.66/Sm 5.5/Chol 5.5), but unacceptable for high-concentration formulations (such as, Van 60/Sm 10/12 Chol 10). Thus, the ultracentrifugation method was further optimized (Method EE-B).

### I. Original Method (Method EE-A, Suitable for Low-Concentration Formulations)

Firstly, an Amicon® Ultra-0.5 Centrifugal Filter (Amicon® filter) with 100 KDa molecular weight cut off (MWCO) was inserted into one microcentrifuge tube. 500 μL of deionized water was added into the Amicon® filter, and the filter in the microcentrifuge tube was centrifuged at 14,000×g for 10 minutes at 4° C. to wash the filter.

500 μL of the liposomal vancomycin suspension (either before lyophilization or after water-reconstitution) was added into the Amicon® filter, and centrifuged at 14,000×g for 20 minutes at 4° C.

About 100 mg of the filtrate (containing free form vancomycin) was transferred into a 10 mL volumetric flask, and deionized water was added to volume (10 mL in total) and mixed well to obtain a diluted filtrate. 2 mL of the diluted filtrate was transferred into a 20 mL volumetric flask, and deionized water was added to volume (20 mL in total) and mixed well to obtain a sample solution for HPLC.

The sample solution for HPLC was assayed via the HPLC-UV method as described in above-mentioned total vancomycin assay to obtain the concentration of free form vancomycin (C_{free}) of the liposomal vancomycin suspension, except the mobile phase was a gradient program as described in Table 2 (Mobile Phase A: ACN/MeOH/solution A=Mar. 4, 1993; Mobile Phase B: ACN/MeOH/solution A=10/40/50; solution A: 7 g/L Tris Buffer, pH 8.05). After that, encapsulation efficiency (EE, in %) was calculated by using the following equation:

$$E E = \frac{{C}_{total} - {C}_{free}}{{C}_{total}} \times 1 \; 00 \% .$$

TABLE 2

| Time | Flow Rate | Mobile Phase A | Mobile Phase B |
|---|---|---|---|
| (min) | (mL/min) | (%) | (%) |
| 0 | 1.0 | 80 | 20 |
| 15 | 1.0 | 80 | 20 |
| 23 | 1.0 | 30 | 70 |
| 25 | 1.0 | 80 | 20 |
| 35 | 1.0 | 80 | 20 |

### II. Optimized Method (Method EE-B, Suitable for High-Concentration Formulations)

Firstly, an Amicon® Ultra-0.5 Centrifugal Filter with 100 KDa molecular weight cut off (MWCO) was washed as described in Method EE-A.

Before centrifugation, the liposomal vancomycin suspension was 10× diluted: 500 mg of the liposomal vancomycin suspension (either before lyophilization or after water-reconstitution) was added into a 5 mL volumetric flask, and deionized water was added to volume (5 mL in total) and mixed well to obtain a diluted liposomal vancomycin suspension. 500 μL of the diluted liposomal vancomycin suspension was transferred into the Amicon® filter, and the filter was centrifuged at 14,000×g for 20 minutes at 4° C. The filtrate was transferred into a 5 mL volumetric flask.

After the above centrifugation, 500 μL of deionized water was added into the same Amicon® filter to wash the liposomal vancomycin remaining in the filter, and the filter was centrifuged at 14,000×g for 20 minutes at 4° C. This washing step was repeated twice, and all filtrate (containing free form vancomycin) was collected in the same 5 mL volumetric flask. Then, deionized water was added to volume (5 mL in total) and mixed well to obtain a sample solution for HPLC.

In the following Table 3, the encapsulating efficiency of the high-concentration formulations prepared by Method I was determined by a semi-optimized method (Method EE-B′), which was the same with the optimized method (Method EE-B) except the liposomal vancomycin suspension was 100× diluted, and no washing step was carried out.

The sample solution for HPLC was assayed via the HPLC-UV method and encapsulation efficiency (EE, in %) was calculated as described in Method EE-A.

The properties of liposomal vancomycin prepared according to Preparation Examples 1 and 2 are listed in the following Table 3.

TABLE 3
Formulation and Properties of Liposomal Vancomycin Suspensions

| Preparation Method of | | | | | |
|---|---|---|---|---|---|
| Liposomal Vancomycin | | Method I | | Method II | |
| Formulation | Van (mg/mL) | 0.66 | 60.0 | 0.66 | 60 |
| | 1^{st} liposome | 5.5/5.5 | 10/10 | 5.5/5.5 | 10/10 |
| | Sm/Chol | | | | |
| | (mg/mL) | | | | |
| Liposomal | PSD (Z-average size | — | — | 131.8 | — |
| Vancomycin | (before 1 yo.) | | | | |
| Suspension | PSD (Z-average size | 117.9 | 109.8 | 116.9 | 117.7 |
| | (reconstituted) | | | | |
| | PDI (before 1 yo.) | — | — | 0.061 | — |
| | PDI (reconstituted) | 0.109 | 0.076 | 0.070 | 0.109 |
| | EE (%) (before 1 yo.) | — | — | 9.0 | — |
| | EE (%) (reconstituted) | 9.0 | 10.3 | 8.9 | 17.7 |
| | | (Method EE-A) | (Method EE-B′) | (Method EE-A) | (Method EE-A) |
| | | | | | 6.1 |
| | | | | | (Method EE-B) |
| | Total Van (mg/mL) | 0.64 | 64.6 | 0.55 | 59.6 |
| | Sm/Chol (mg/mL) | 5.06/5.16 | N/A | 5.80/5.40 | 10.7/10.3 |
| | pH | about 6.0 | 3.98 | 6.1 | 6.2 |

From Table 3, it is found that the liposomal vancomycin suspension before lyophilization (before 1yo.) or after lyophilization and then water-reconstitution (reconstituted) made by either method of Preparation Example 1 (Method I) or Preparation Example 2 (Method II) have similar properties.

In comparison, the liposomal vancomycin suspension prepared by Method II before lyophilization (before 1yo.) and after lyophilization and then water-reconstitution (reconstituted) had similar EE % (9.0% vs. 8.9%) and minor PSD reduction (from 131.8 nm to 116.9 nm). The results suggested that the liposomal vancomycin suspension can be lyophilized for long-term storage and reconstituted before use.

The EE % after lyophilization and then water-reconstitution (reconstituted) of the liposomal vancomycin suspension made by Method II was detected by the original method (Method EE-A) and optimized method (Method EE-B), and the results listed in Table 3 indicated that serious error might occur when the high-concentration formulation was examined by the original method. Another formulation, (liposomal vancomycin 5A: Van 66/Sm 11/Chol 11) also had similar results-22.9% by Method EE-A and 7.4% by Method EE-B. The high-concentration formulations should be (1) diluted before ultrafiltration by Amicon® filter, and (2) further washed with deionized water twice to improve the vancomycin recovery rate of this EE method.

After further studies, it was found that the EE % of the vancomycin pharmaceutical composition of the present invention can be determined by Method EE-B but the liposomal vancomycin suspension was 100× diluted. In other words, the liposomal vancomycin suspension was 100× diluted: 500 mg of the liposomal vancomycin suspension (either before lyophilization or after water-reconstitution) was added into a 50 mL volumetric flask, and deionized water was added to volume (50 mL in total) and mixed well to obtain a diluted liposomal vancomycin suspension. 500 μL of the diluted liposomal vancomycin suspension was transferred into the Amicon® filter, and the filter was centrifuged at 14,000×g for 20 minutes at 4° C. The filtrate was transferred into a 5 mL volumetric flask. After the above centrifugation, 500 μL of deionized water was added into the same Amicon® filter to wash the liposomal vancomycin remaining in the filter, and the filter was centrifuged at 14,000×g for 20 minutes at 4° C. This washing step was repeated twice, and all filtrate (containing free form vancomycin) was collected in the same 5 mL volumetric flask. Then, deionized water was added to volume (5 mL in total) and mixed well to obtain a sample solution for HPLC.

### Preparation Example 3: Preparation of Second Liposome Suspension

An appropriate amount of a lipid was fully dissolved in ethanol (EtOH) at ambient temperature to obtain a “lipid phase solution of second liposome”. The lipid could be sphingomyelin (Sm); or a mixture of 90 wt % or more of Sm and 10 wt % or less of another lipid selected from cholesterol, phospholipids, phospholipid derivatives, or a combination thereof. Next, the lipid phase solution of second liposome was added into water to have a final EtOH concentration at around 9 wt % to 15 wt % and form a crude second liposome suspension. The crude second liposome suspension was subjected to repeated extrusions through a series of polycarbonate track-etched membranes at 50° C. to 60° C. and an elevated pressure to obtain a second liposome suspension (i.e., second liposome in a liquid suspension form) to obtain a second liposome suspension. After the extrusion, the Z-average size of the second liposome suspension should be around 50 nm to 70 nm. The second liposome suspension could be used directly; or, the second liposome suspension could potentially be lyophilized.

Alternatively, the preparation of the second liposome suspension could also be carried out by, but not limited to, processes such as microfluidics, and the sizing could be achieved by methods such as ultrasonication or high-pressure homogenization.

In addition, the PSD stability of the second liposome suspension after extrusion may be affected by the EtOH content remaining in the solution. For buffer exchange and concentration, diafiltration (or dialysis or buffer exchange) can be adopted by using KrosFlo® KR2i TFF System with a 100 kDa mPES hollow fiber. Other equipment based on the same dialysis principle could be used.

It was found that when the lipid was dissolved in EtOH at low concentration (e.g., 10 mg/mL to 50 mg/mL lipid in EtOH) and added into water to obtain a crude second liposome suspension (with a final ethanol concentration at 9 wt % to 15 wt %, and a lipid concentration of 1 mg/mL to 2 mg/mL), the resulting second liposome suspension had a Z-average size of 45 nm to 90 nm. After concentration, it could be used directly without any extrusion.

### Example 1A: Liposomal Vancomycin 1A

The liposomal vancomycin 1A was prepared according to Preparation Example 1 (Method I) using 2 mg/mL vancomycin HCl in histidine-sucrose buffer solution as the API solution, and 50 mg/mL Sm and 50 mg/mL Chol dissolved in CHCl₃ as the lipid phase solution of first liposome to make the liposomal vancomycin comprising 10 mg/mL total lipid. The weight ratio of vancomycin to the first liposome is 0.2:1; and the liposomal vancomycin suspension was subjected to 11 repeated extrusions through polycarbonate track-etched membranes having pore size of 0.2 μm. The resulting liposomal vancomycin 1A had a Van concentration of 2 mg/mL, a Sm concentration of 5 mg/mL, a Chol concentration of 5 mg/mL, a PSD (Z-average) of 190 nm, and an EE of 12.2%.

### Example 2A: Liposomal Vancomycin 2A

The liposomal vancomycin 2A was prepared by the method of Example 1A except using 50 mg/mL vancomycin HCl in histidine-sucrose buffer solution as the API solution, and 150 mg/mL Sm and 150 mg/mL Chol dissolved in CHCl₃ as the lipid phase solution of first liposome to make the liposomal vancomycin comprising 30 mg/mL total lipid; and the free form Vancomycin was further reduced by PD MidiTrap G-25 size exclusion columns (from Cytiva) per manufacturer's instructions. The resulting liposomal vancomycin 2A had a Van concentration of 2 mg/mL, a Sm concentration of 5 mg/mL, a Chol concentration of 5 mg/mL, a PSD (Z-average) of 190 nm, and an EE of 40%.

### Example 3A: Liposomal Vancomycin 3A

The liposomal vancomycin 3A was prepared by the method of Example 1A except using 0.6 mg/mL vancomycin HCl in histidine-sucrose buffer solution as the API solution, and 50 mg/mL Sm and 50 mg/mL Chol dissolved in CHCl₃ as the lipid phase solution of first liposome to make the liposomal vancomycin comprising 10 mg/mL total lipid; and the weight ratio of vancomycin to the first liposome is 0.06:1. The resulting liposomal vancomycin 3A had a Van concentration of 0.6 mg/mL, a Sm concentration of 5 mg/mL, a Chol concentration of 5 mg/mL, a PSD (Z-average) of 190 nm, and an EE of 14.6%.

### Example 4A: Liposomal Vancomycin 4A

The liposomal vancomycin 4A was prepared by the method of Example 1A except using 0.6 mg/mL vancomycin HCl in histidine-sucrose buffer solution as the API solution, and 50 mg/mL Sm and 50 mg/mL Chol dissolved in the CHCl₃ as the lipid phase solution of first liposome to make the liposomal vancomycin comprising 10 mg/mL total lipid, and the liposomal vancomycin suspension was subjected to 5 repeated extrusions through polycarbonate track-etched membranes having pore size of 0.2 μm and 11 repeated extrusions through polycarbonate track-etched membranes having pore size of 0.1 μm. The resulting liposomal vancomycin 4A had a Van concentration of 0.6 mg/mL, a Sm concentration of 5 mg/mL, a Chol concentration of 5 mg/mL, a PSD (Z-average) around 130 nm, and an EE of 9.7% or 9.0% (e.g., in the two preparations of the liposomal vancomycin 4A made for the following animal studies C and D, the values of EE were consistent at 9.7% and 9.0%, respectively).

### Example 5A: Liposomal Vancomycin 5A

The liposomal vancomycin 5A was prepared according to Preparation Example 2 (Method II) using 66 mg/mL vancomycin HCl in histidine-sucrose buffer solution as the API solution, and 110 mg/mL Sm and 110 mg/mL Chol in dissolved in CHCl₃ as the lipid phase solution of first liposome to make the liposomal vancomycin comprising 22 mg/mL total lipid. The weight ratio of Vancomycin to the first liposome is 3:1; and the liposomal vancomycin suspension was subjected to 15 repeated extrusions through polycarbonate track-etched membranes having pore size of 0.1 μm. The resulting liposomal vancomycin 5A had a Van concentration of 66 mg/mL, a Sm concentration of 11 mg/mL, a Chol concentration of 11 mg/mL, a PSD (Z-average) of 118.6 nm, and an EE of 7.4%.

### Example 6A: Liposomal Vancomycin 6A

The liposomal vancomycin 6A was prepared by the method of Example 5A, except that the liposomal vancomycin suspension was subjected to 5 repeated extrusions through polycarbonate track-etched membranes having pore size of 0.2 μm and 4 repeated extrusions through polycarbonate track-etched membranes having pore size of 0.1 μm. The resulting liposomal vancomycin 6A had a Van concentration of 66 mg/mL, a Sm concentration of 11 mg/mL, a Chol concentration of 11 mg/mL, a PSD (Z-average) of 116.3 nm, and an EE of 11.2%.

### Example 1B: Second Liposome Suspension 1B

The second liposome suspension 1B was prepared according to Preparation Example 3, wherein the lipid dissolved in ethanol was 100% sphingomyelin (Sm) at around 100 mg/mL, the lipid phase solution of second liposome was added into water to have a final EtOH concentration at 10 wt %, and the crude second liposome suspension was subjected to 11 repeated extrusions through polycarbonate track-etched membranes having pore size of 0.1 μm. The resulting second liposome suspension 1B had a Sm concentration of 10 mg/mL, and a PSD (Z-average) of 120 nm.

### Example 2B: Second Liposome Suspension 2B

The second liposome suspension 2B was prepared by the method of Example 1B, except that the lipid dissolved in ethanol was at a concentration of 20 mg/mL, and the resulting second liposome suspension had a concentration of about 2 mg/mL (with a final EtOH concentration at about 10 wt %). After further concentration by evaporation at 55° C. and concentration adjustment, the resulting second liposome suspension 2B had a Sm concentration of 10 mg/mL, and a PSD (Z-average) of 76 nm.

### Example 3B: Second Liposome Suspension 3B

The second liposome suspension 3B was prepared by the method of Example 2B, except that the lipid dissolved in ethanol was at a concentration of 10 mg/mL, and the resulting second liposome suspension had a concentration of about 1 mg/mL. After further concentration by evaporation at 55° C. and concentration adjustment, the resulting second liposome suspension 3B had a Sm concentration of 10 mg/mL, and a PSD (Z-average) of 47 nm.

### Example 4B: Second Liposome Suspension 4B

The second liposome suspension 4B was prepared by the method of Preparation Example 3, wherein the lipid dissolved in ethanol was 100% sphingomyelin (Sm) at around 125 mg/mL, the lipid phase solution of second liposome was added into water to have a final EtOH concentration at 10 wt %, and the crude second liposome suspension was subjected to 25 repeated extrusions through polycarbonate track-etched membranes having pore size of 0.05 μm. After diafiltration using KrosFlo® KR2i TFF System and final concentration adjustment, the resulting second liposome suspension 4B had a Sm concentration of 10 mg/mL, and a PSD (Z-average) of 71.1 nm.

### Example 5B: Second Liposome Suspension 5B

The second liposome suspension 5B was prepared by the method of Example 4B, except that 0.3% NaCl was additionally added into the second liposome suspension. After concentration adjustment, the resulting second liposome suspension 5B had a Sm concentration of 10 mg/mL, and a PSD (Z-average) of 72.4 nm.

### Example 6B: Second Liposome Suspension 6B

The second liposome suspension 6B was prepared by the method of Preparation Example 3, wherein the lipid dissolved in ethanol was 100% sphingomyelin (Sm) at around 125 mg/mL, the lipid phase solution of second liposome was added into water to have a final EtOH concentration at 10 wt %, and the crude second liposome suspension was subjected to 7 repeated extrusions through polycarbonate track-etched membranes having pore size of 0.1 μm and 8 repeated extrusions through polycarbonate track-etched membranes having pore size of 0.05 μm, and 0.3% NaCl was additionally added into the second liposome suspension. After diafiltration using KrosFlo® KR2i TFF System and final concentration adjustment, the resulting second liposome suspension 6B had a Sm concentration of 10 mg/mL, and a PSD (Z-average) of 69.8 nm.

### Preparation Example 4: Reconstitution of Lyophilized Liposomal Vancomycin with Second Liposome Suspension to Form the Vancomycin Pharmaceutical Composition

To ensure proper liposome formation, uniformity, and stability, the reconstitution of a lyophilized liposomal composition generally performs at a temperature higher than the phase-transition temperature (Tₘ) of the lyophilized liposomal composition itself.

However, a warming up step required for the diluent or reconstitution liquid is not convenient for clinical practice. It is therefore preferred to develop a lyophilized pharmaceutical drug product that could be reconstituted at ambient temperature.

In the present invention, the second liposome suspension (in a liquid suspension form) could be used to reconstitute the lyophilized liposomal vancomycin (in solid form). The Z-average size and EE of lyophilized liposomal vancomycin reconstituted with (1) pre-warmed (50° C. to 60° C.) second liposome (in a liquid suspension form), and (2) second liposome (in a liquid suspension form) at ambient temperature were compared, and both the Z-average size and EE results had no significant difference (data not shown).

Animal Test 1: Anti-Bacterial Efficacy Testing in S. aureus USA 300 MRSA (BAA-1556), Mouse Bacteremia LD90-100 Model

To evaluate the overall anti-bacterial efficacy of the vancomycin pharmaceutical composition of the present invention, the S. aureus USA 300 MRSA (BAA-1556), Mouse Bacteremia LD90-100 Model established at Eurofins Panlabs Discovery Services Taiwan was utilized. This model evaluates the antimicrobial efficacy of test compound or composition for treatment of systemic infections with community acquired MRSA.

Organism: S. aureus USA300, originally named FPR 3757, is a community-associated MRSA that emerged as an epidemic strain and causes rapidly progressive and fatal diseases. S. aureus USA300 is a multiple drug resistant (MDR) organism, which is resistant to mupirocin, quinolones, macrolides and all classes of β-lactam antibiotics.

Procedure: Groups of 10 female BALB/c mice weighing 18±2 grams (g) were used in the test. Each animal was inoculated with an intravenously administered LD90-100 dose (1.0×10⁸ to 2.0×10⁸ colony-forming units (CFU) per mouse, CFU/mouse) of Methicillin Resistant Staphylococcus aureus (S. aureus USA300 MRSA, BAA-1556) suspended in 0.1 mL phosphate buffered saline (PBS). Vehicle for each study per design as control, test compound or composition was then administered at designated time points via intravenous (IV) administration. Mortality was recorded daily during the following 10 days. Prevention of mortality was recorded in percentage. The following tests were carried out by the LD90-100 model. The results of survival rates over 10.5 days were obtained. A higher survival rate after a certain amount of time indicates a potentially better anti-bacteria efficacy for that period of time.

### (1) Animal Study A

To study whether a higher EE of the liposomal vancomycin is desired, five groups of mice were IV administered with the following test compound or composition once, and the first administration was provided at 2 h post infection:

A1: Free Van HCl, Van 100 mg/kg of body weight (as positive control);

A2: Free Van HCl, Van 10 mg/kg of body weight;

A3: Vancomycin pharmaceutical composition with a low EE (12.2%), comprising liposomal vancomycin 1A and second liposome suspension 1B at a lipid weight ratio of 1:1, Van 10 mg/kg of body weight;

A4: Vancomycin pharmaceutical composition with a high EE (40%), comprising liposomal vancomycin 2A and second liposome suspension 1B at a lipid weight ratio of 1:1, Van 10 mg/kg of body weight; and

A5: Saline (as quality control).

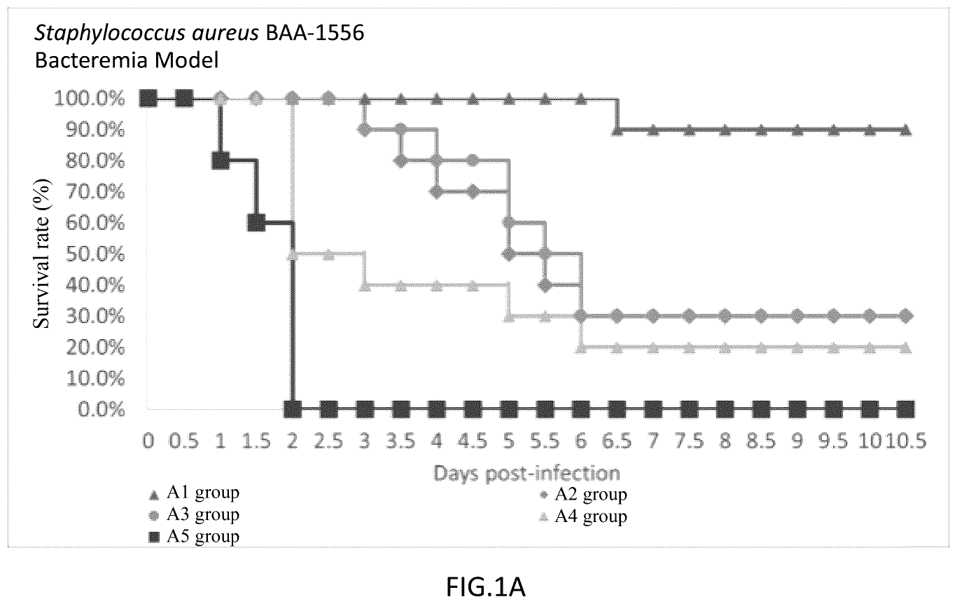

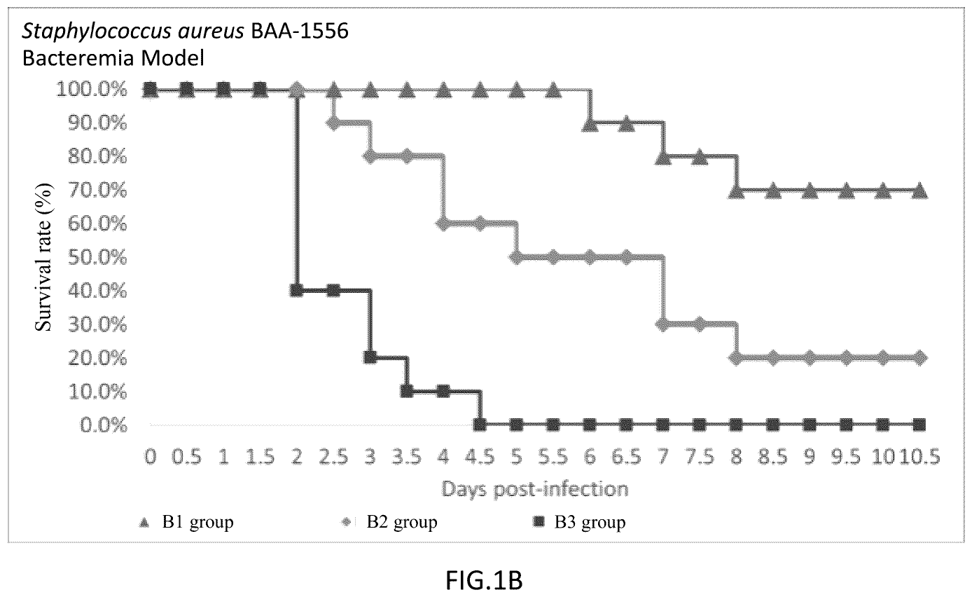

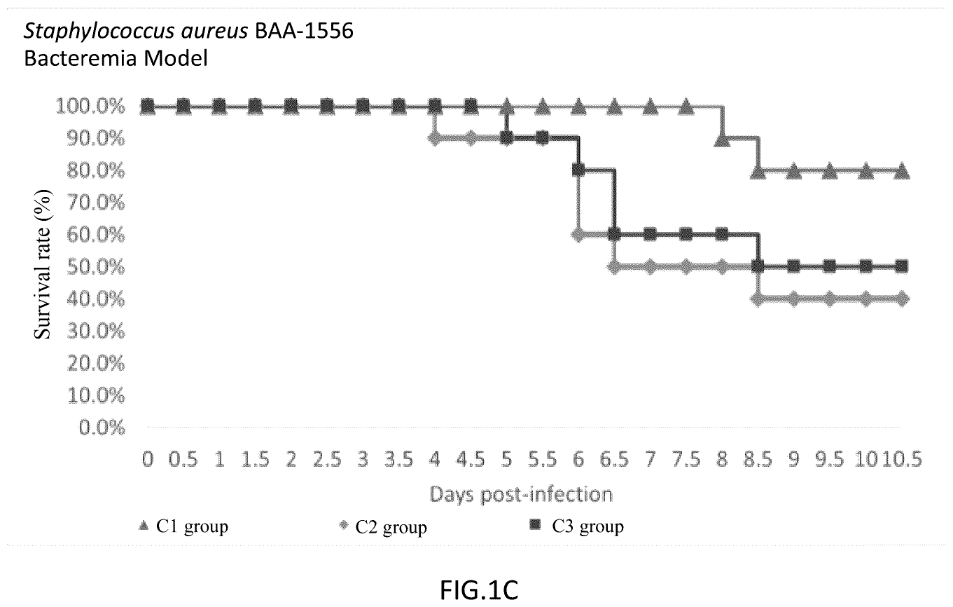

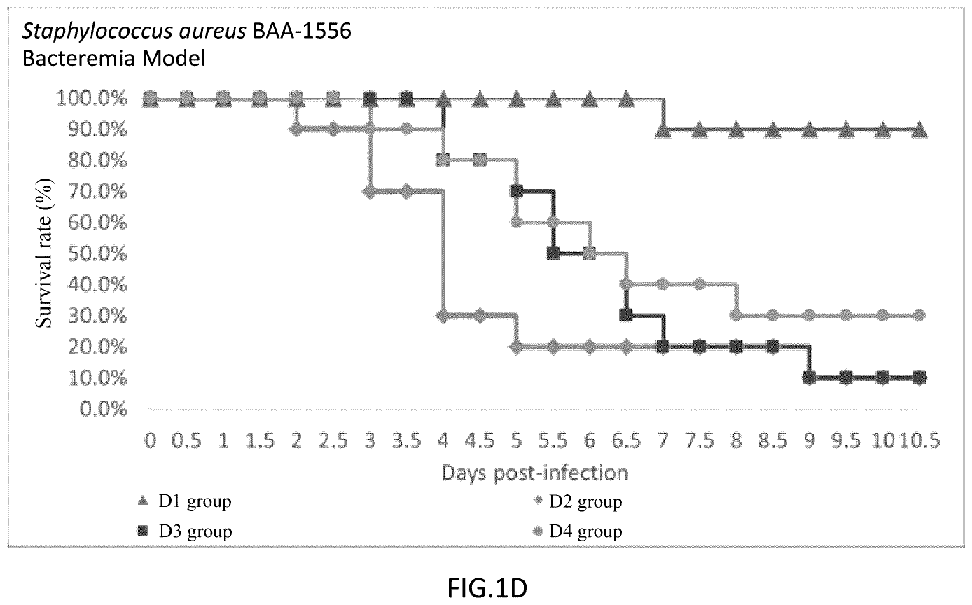

The survival rates of A1 to A5 groups in this animal study are shown in FIG. 1A. Half of the mice died early on Day 2 for A4 group (with a high EE). This suggests that despite the potential advantages offered by the vancomycin pharmaceutical composition, such as prolonged pharmacokinetics (PK), altered biodistribution, and the toxin-sequestering activity from the liposome composition, the presence of free vancomycin in the bloodstream remains critically important for rapidly killing bacteria during bacteremia. Therefore, a lower EE range of the vancomycin pharmaceutical composition might be optimal for the effective treatment of bacteremia. Notably, this concept deviates from the paradigm of most liposomal drugs, where a high EE (often exceeding 95% or 99%) is typically preferred.

In comparison with free Van HCl at equal dose (10 mg/kg), it seems that A3 group (with a low EE) provides a balance between liposomal encapsulation while sufficient free vancomycin is kept for instant killing.

### (2) Animal Study B

In comparison with the Animal Study A, the administration regimen was tuned from once (2 h post infection) to three times (2 h, 6 h, and 24 h post infection), and the dose was reduced to 30% for each dose in Animal Study B. It was expected that the divided doses can provide better vancomycin plasma concentration time over minimum inhibitory concentration (MIC) coverage. This regimen should mimic clinical vancomycin clinical practice better. In addition, the second liposome was not added in Animal Study B.

In this animal study, three groups of mice were IV administered with the following test compound or composition three times at 2 h, 6 h and 24 h post infection:

B1: Free Van HCl, Van 30 mg/kg of body weight (as positive control);

B2: Free Van HCl, Van 3 mg/kg of body weight; and

B3: Vancomycin pharmaceutical composition with a high Z-average size (190 nm), comprising liposomal vancomycin 3A only, Van 3 mg/kg of body weight.

The survival rates of B1 to B3 groups in this animal study are shown in FIG. 1B. Generally, the modified regimen of the Animal Study B provided a similar model performance with the Animal Study A that the low dose free Van HCl group (i.e., B2 group, free Van HCl, 3 mg/kg) provided approximately 20% survival rate at the end of the study period. As a result, this protocol (three IV injections at 2 h, 6 h and 24 h post infection with high dose free Van HCl 30 mg/kg and low dose free Van HCl 3 mg/kg) was also used for the following Animal Study C and Animal Study D.

In comparison with B2 group, it seems that the survival curve of B3 group is worse than the B2 group having the same dose level of free Van HCl. This may be because high Z-average size is not beneficial for prolonged circulation, and most of the 14.6% encapsulated vancomycin was probably sequestered rapidly by the mononuclear phagocyte system (MPS), thus not bioavailable for combating bacteremia, leading to the poor therapeutic performance.

### (3) Animal Study C

Following the Animal Study B, the vancomycin pharmaceutical composition with a lower Z-average size (130 nm) was dosed for comparison. The second liposome was still not added in Animal Study C, same as Animal Study B.

In this animal study, three groups of mice were IV administered three times at 2 h, 6 h and 24 h post infection:

C1: Free Van HCl, Van 30 mg/kg of body weight (as positive control);

C2: Free Van HCl, Van 3 mg/kg of body weight; and

C3: Vancomycin pharmaceutical composition with low Z-average size (130 nm), comprising liposomal vancomycin 4A only, Van 3 mg/kg of body weight.

The survival curve obtained from Animal Study C was shown in FIG. 1C. It indicated that the C3 group provided a slightly better survival curve than the C2 group. According to the cross-comparison of the results from Animal Study B where B3 group performed much worse than B2 group (B2 and C2 groups were the same and could be used for bridging), we could conclude that Z-average size at 130 for liposomal vancomycin suspension is preferred for not only better physical stability but also better in vivo anti-bacterial performance.

### (4) Animal Study D

Following the Animal Study C which confirms the preferred Z-average size of the liposomal vancomycin suspension (130 nm), the second liposome was added back in Animal Study D to see if the second liposome can further boost the therapeutic performance and improve the survival curve.

In this animal study, four groups of mice were IV administered with the following test compound or composition three times at 2 h, 6 h and 24 h post infection:

D1: Free Van HCl, Van 30 mg/kg of body weight (as positive control);

D2: Free Van HCl, Van 3 mg/kg of body weight;

D3: Vancomycin pharmaceutical composition comprising liposomal vancomycin 4A and second liposome suspension 2B (with high Z-average size at 76 nm) at a lipid weight ratio of 1:1, Van 3 mg/kg of body weight; and

D4: Vancomycin pharmaceutical composition comprising liposomal vancomycin 4A and second liposome suspension 3B (with low Z-average size at 47 nm) at a lipid weight ratio of 1:1, Van 3 mg/kg of body weight.

The survival rates of D1 to D4 groups in this animal study are shown in FIG. 1D. The survival rate differences between D2 and D3 groups (D2 vs. D3) and D2 and D4 groups (D2 vs. D4) from Day 3 to Day 10 are calculated and compared. In accordance with Log-rank test, the survival rate differences of D2 vs. D3 and D2 vs. D4 on Day 4 and Day 5 are statistically significant (p-values are listed in Table 4, * represents p<0.05), suggesting an enhanced anti-bacterial efficiency in comparison with free Van HCl at equal dose.

TABLE 4
p-values (by Log-rank test)

| | Day 3 | Day 4 | Day 5 | Day 6 |
|---|---|---|---|---|
| D2 vs. D3 | 0.0675 | 0.0188* | 0.0141* | 0.0450* |
| D2 vs. D4 | 0.2593 | 0.0320* | 0.0424* | 0.0730 |
| | Day 7 | Day 8 | Day 9 | Day 10 |
| D2 vs. D3 | 0.1807 | 0.1807 | 0.1807 | 0.2058 |
| D2 vs. D4 | 0.0730 | 0.1215 | 0.1961 | 0.1139 |

In comparison of Animal Study D with Animal Study C, it is clear that the addition of the second liposome (both D3 and D4 groups) improves anti-bacteria efficacy.

### Animal Test 2: Pharmacokinetic Study of Reconstituted Liposomal Vancomycin in Mice

Among several potential benefits (such as reduced toxicity, controlled release, protection from degradation, enhanced stability, improved pharmacokinetics (PK), targeted drug delivery, etc.) of liposome encapsulation of drugs for parenteral usage, the fundamental and probably the most important benefit as a proof-of-concept one for non-clinical study is its PK compared with its native free form drugs.

In this test, two groups of female ICR mice (body weight: about 30 g, 3 mice for each group) were slow IV (about 30 seconds) administered with the following test compound or composition. Blood samples were collected at specific time points for examination of total vancomycin exposure levels in all plasma samples derived from the blood samples:

E1: Free Van HCl, Van 150 mg/kg of body weight; and

E2: Vancomycin pharmaceutical composition, comprising liposomal vancomycin 5A and second liposome suspension 4B at a lipid weight ratio of 1:1; 150 mg/kg of body weight.

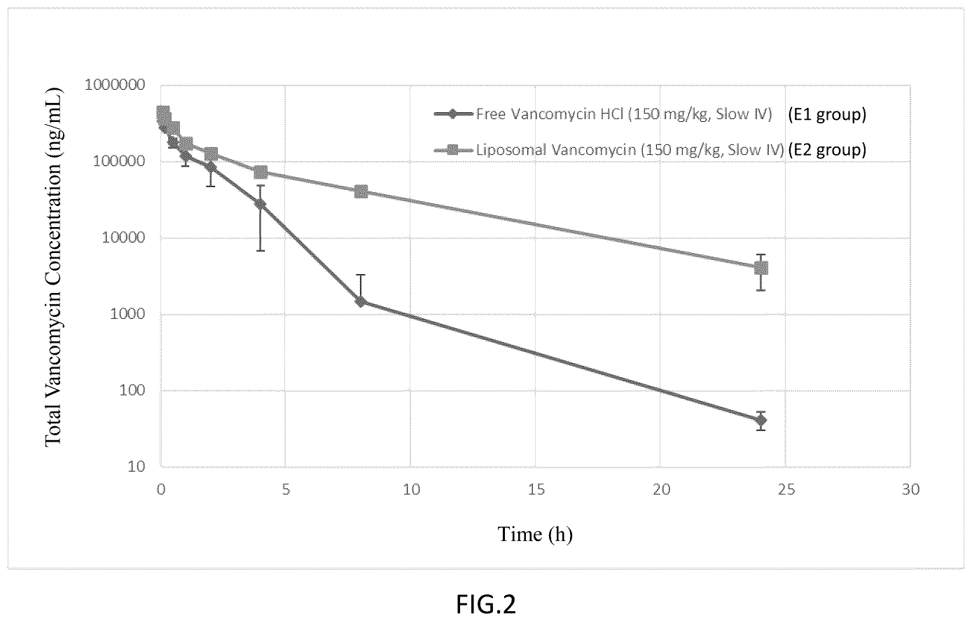

The plasma concentrations of E1 and E2 groups of this test are shown in FIG. 2; and the exposure levels of vancomycin in mice plasma samples of E1 and E2 groups were recorded in the Tables 4 and 5, respectively. In this study, the levels of vancomycin are the levels of “total vancomycin”, including liposomal vancomycin (LipoVan) and free form vancomycin (free Van).

TABLE 5
Vancomycin exposure level of plasma samples of mice in E2 group

| | Sample concentration (ng/ml) | | | | |
|---|---|---|---|---|---|
| Time (h) | Mouse#1 | Mouse#2 | Mouse#3 | Mean | SD |
| 0.083 | 350312 | 299741 | 347261 | 332438 | 28357 |
| 0.167 | 280058 | 275621 | 266805 | 274161 | 6746 |
| 0.5 | 153968 | 170626 | 202720 | 175771 | 24780 |
| 1 | 85226 | 148901 | 119948 | 118025 | 31881 |
| 2 | 45419 | 87009 | 122604 | 85011 | 38631 |
| 4 | 7850 | 25226 | 49257 | 27444 | 20792 |
| 8 | 195 | 671 | 3539 | 1468 | 1809 |
| 24 | 33 | 37 | 53 | 41 | 11 |

TABLE 6
Vancomycin exposure level of plasma samples of mice in E2 group

| | Sample concentration (ng/mL) | | | | |
|---|---|---|---|---|---|
| Time (h) | Mouse#1 | Mouse#2 | Mouse#3 | Mean | SD |
| 0.083 | 432298 | 466885 | 417020 | 438734 | 25548 |
| 0.167 | 370143 | 391790 | 348001 | 369978 | 21895 |
| 0.5 | 254141 | 305651 | 277565 | 279119 | 25790 |
| 1 | 173753 | 180845 | 167553 | 174050 | 6651 |
| 2 | 125239 | 122570 | 130992 | 126267 | 4304 |
| 4 | 73701 | 78143 | 67787 | 73210 | 5195 |
| 8 | 39797 | 39554 | 42130 | 40494 | 1422 |
| 24 | 2171 | 6177 | 3791 | 4046 | 2015 |

In addition, the PK parameters of vancomycin in mice plasma samples of E1 and E2 groups were recorded in Tables 7 and 8, respectively. The pharmacokinetics (PK) parameters listed in Tables 7 and 8 include:
1. t_{1/2}: Apparent terminal elimination half-life;
2. C₀: Initial concentration;
3. AUClast: Area under the plasma concentration-time curve (AUC) from the time of dosing to the time of the last measurable concentration;
4. AUCInf: AUC from time of dosing extrapolated to infinity;
5. AUC/D: AUC divided by dose;
6. AUC Extr: AUC extrapolated from last time point to infinity as a percentage of total AUC;
7. MRT: The average time a drug molecule stays in the body. It's calculated by summing the total time each molecule spends in the body and dividing by the number of molecules.
8. Vss: Apparent volume of distribution in steady state; and
9. CL: Apparent total body clearance of the drug from plasma.

TABLE 7
PK parameters of El group

| | Sample concentration (ng/ml) | | | | |
|---|---|---|---|---|---|
| | Mouse#1 | Mouse#2 | Mouse#3 | Mean | SD |
| t_{1/2} (h) | 1.80 | 1.81 | 1.95 | 1.85 | 0.08 |
| C₀ (ng/mL) | 437024 | 325646 | 450563 | 404411 | 68547 |
| AUCₗₐₛₜ (h*ng/mL) | 327719 | 491949 | 645208 | 488292 | 158776 |
| AUC_{Inf} (h*ng/mL) | 327805 | 492045 | 645357 | 488402 | 158808 |
| AUC/D (h*kg*ng/mL/mg) | 2185 | 3280 | 4302 | 3256 | 1059 |
| AUC Extr (%) | 0.03 | 0.02 | 0.02 | 0.02 | 0.00 |
| MRT (h) | 1.11 | 1.60 | 2.17 | 1.62 | 0.53 |
| Vss (L/kg) | 0.51 | 0.49 | 0.50 | 0.50 | 0.01 |
| CL (mL/min/kg) | 7.63 | 5.08 | 3.87 | 5.53 | 1.92 |

TABLE 8
PK parameters of E2 group

| | Sample concentration (ng/mL) | | | | | Ratio of |
|---|---|---|---|---|---|---|
| | Mouse | Mouse | Mouse | | | LipoVan/ |
| | #1 | #2 | #3 | Mean | SD | FreeVan |
| t_{1/2} | 3.90 | 5.60 | 4.75 | 4.75 | 0.85 | 2.57 |
| (h) | | | | | | |
| C₀ | 503958 | 555213 | 498652 | 519275 | 31237 | 1.28 |
| (ng/mL) | | | | | | |
| AUCₗₐₛₜ | 1194650 | 1269892 | 1220821 | 1228454 | 38197 | 2.52 |
| (h*ng/mL) | | | | | | |
| AUC_{Inf} | 1206859 | 1319791 | 1246788 | 1257812 | 57268 | 2.58 |
| (h*ng/mL) | | | | | | |
| AUC/D | 8046 | 8799 | 8312 | 8385 | 382 | 2.58 |
| (h*kg*ng/mL/mg) | | | | | | |
| AUC Extr | 1.01 | 3.78 | 2.08 | 2.29 | 1.40 | 100.05 |
| (%) | | | | | | |
| MRT | 4.49 | 5.66 | 5.06 | 5.07 | 0.58 | 3.12 |
| (h) | | | | | | |
| Vss | 0.56 | 0.64 | 0.61 | 0.60 | 0.04 | 1.21 |
| (L/kg) | | | | | | |
| CL | 2.07 | 1.89 | 2.01 | 1.99 | 0.09 | 0.36 |
| (mL/min/kg) | | | | | | |

It appears that the vancomycin pharmaceutical composition of the E2 group demonstrated improved PK parameters. In comparison with the E1 group, the t_{1/2} increased from 1.85 h to 4.75 h; and Co and AUCinf increased to 1.28× and 2.58×, respectively. A significantly prolonged circulation attributed to vancomycin pharmaceutical composition was also clearly demonstrated (also see FIG. 2).

Animal Test 3: Efficacy Testing in Staphylococcus aureus (VRSA and VRS-2), Mouse Infected Thigh Model

For efficacy testing in muscle tissue infections, a mouse infected thigh model has been described in the literature as a tissue infection model for vancomycin resistant MRSA. The model established by Eurofins PDS Taiwan is described as below:

Organism: S. aureus strain VRS-2, also known as VRSA strain Hershey, is a Van-A Type S. aureus . . . . It is also a multiple drug resistant (MDR) organism, which is resistant to methicillin, carbapenems, cephalosporins, penicillins, vancomycin (MIC>64), quinolones (including levofloxacin (LVX) and ciprofloxacin (CIP)), macrolides (including erythromycin (ERY) and clindamycin (CLI)) and trimethoprim sulfamethoxazole.

Procedure: Groups of 5 female specific-pathogen-free ICR mice weighing 22±2 g were used. Animals were immunosuppressed by two intraperitoneal injections of cyclophosphamide, the first injection at 150 mg/kg was treated 4 days before infection (day −4) and the second injection at 100 mg/kg was treated 1 day before infection (day −1). On day 0, animals were inoculated intramuscularly (0.1 mL, on thigh) with 1×10⁵ CFU/mouse of the Staphylococcus aureus vancomycin resistant (VRS-2) strain into one thigh. Vehicle (empty liposomes), test compound or compositions were then administered intravenously (IV) (each time for about 30 seconds) twice a day (BID) with a 12 h interval (q12h) at 2 h and 14 h after the inoculation per the instruction of the tissue infection model (Eurofins PDS Taiwan). The volume of the injection solution for each injection was 5 mL/kg of body weight. At 26 hours after inoculation, animals were humanely euthanized with carbon dioxide (CO₂) asphyxiation then the thigh tissue was harvested from each mouse. The removed thigh tissues were respectively homogenized in 3 mL of PBS, pH 7.4, with a polytron homogenizer. 0.1 mL of the resulting homogenates were respectively used for serial 10-fold dilutions to obtain a series of diluted samples, and each diluted sample was plated on a nutrient agar plate for colony count determination to give bacteria count data.

F1: untreated, as the baseline control, in which mice were sacrificed at 2 h after inoculation for initial bacterial counts for thigh tissue at the start of administration;

F2: vehicle (empty liposome, equal lipid amount as F5 group);

F3: Free Van HCl, Van 300 mg/kg of body weight;

F4: Free Van HCl, Van 150 mg/kg of body weight;

F5: Vancomycin pharmaceutical composition, comprising liposomal vancomycin 5A and second liposome suspension 4B at a lipid weight ratio of 1:1, 150 mg/kg of body weight; wherein the second liposome suspension 4B was pre-warmed to 50° C. to 60° C. then used for the reconstitution of liposomal vancomycin 5A; and

F6: Same vancomycin pharmaceutical composition as F5 group, 150 mg/kg of body weight; wherein the second liposome suspension 4B at ambient temperature was used for the reconstitution of liposomal vancomycin 5A.

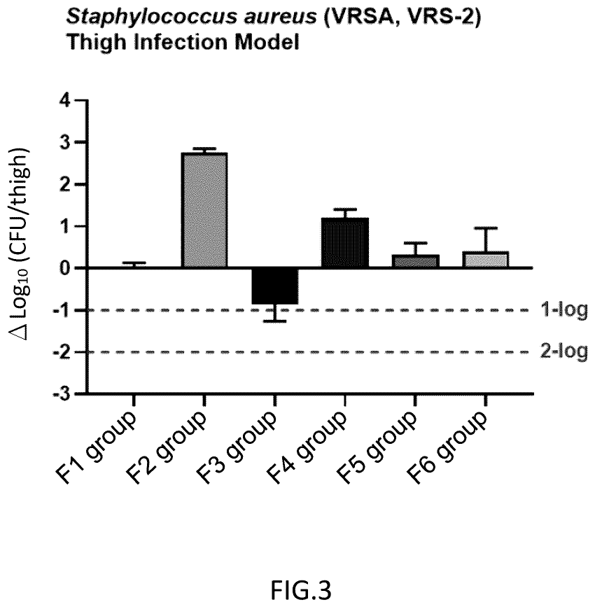

The logarithmic numbers of colony-forming units (CFU) per thigh of F1 to F6 groups are calculated and the difference of the Log₁₀ treatment−Log₁₀ baseline (ΔLog₁₀ (CFU/thigh)) shown in FIG. 3. A lower logarithmic number of colony-forming units indicates a better anti-bacterial performance. The ΔLog₁₀ at 1 indicates that the amount of viable bacteria is the same as untreated baseline control at the time of treatment initiation, suggesting static bacteria growth. The dotted line “1-log” indicates that the amount of viable bacteria is reduced to one tenth (1/10). The dotted line “2-log” indicates that the amount of viable bacteria is reduced to one hundredth (1/100). The anti-bacterial performance of F5 and F6 groups, the vancomycin pharmaceutical compositions reconstituted at two different temperatures, have no obvious difference. The ambient temperature is applicable for the reconstitution of lyophilized liposomal vancomycin by the second liposome.

Animal Test 4: Efficacy Testing in S. aureus (USA300 MRSA (BAA-1556), Mouse Lung Infection Model

For antimicrobial efficacy testing in MRSA lung infection, a mouse infected lung model which has been described in the literature as a tissue infection model with vancomycin resistant MRSA was utilized. The model established by Eurofins PDS Taiwan is described as below:

Organism: S. aureus USA300, originally named FPR 3757, is a community-associated MRSA that emerged as an epidemic strain and causes rapidly progressive and fatal diseases. S. aureus USA300 is a MDR organism, which is resistant to mupirocin, quinolones, macrolides and all classes of β-lactam antibiotics.

Procedure: Groups of 5 female specific-pathogen-free ICR mice weighing 22±2 g were used. Animals were immunosuppressed by two intraperitoneal injections of cyclophosphamide: the first at 150 mg/kg 4 days before infection (day-4) and the second at 100 mg/kg 1 day before infection (day-1). On day 0, animals are anesthetized with pentobarbital (50 mg/kg, IV) then inoculated intranasally (0.05 mL/lung) with 0.5×10⁷ CFU/mouse to 1.0×10⁷ CFU/mouse of exponentially growing Staphylococcus aureus BAA-1556 suspension in PBS containing 3% mucin for infection. Vehicle (normal saline), test compound or composition was then administered 2 and 14 hours later per the specification of the study design table. At 50 h and 74 h post-infection, the animals were euthanized with CO₂ asphyxiation and their lung tissue was harvested from each of the test animals. The removed tissue was then homogenized in 1 mL PBS, pH 7.4, with a polytron homogenizer. After that, 0.1 mL of Homogenates was taken and 10-fold diluted with PBS, pH 7.4 and plated onto a nutrient agar plate for colony count determination to give bacteria count data. In addition, the homogenates were also used for vancomycin assay, for the evaluation of whether lung accumulation was enhanced.

G1: Untreated, as the baseline control, in which mice were sacrificed at 2 h post-infection for initial bacterial counts for lung tissue at the start of administration;

G2: Vehicle (saline); mice were sacrificed at 26 h post-infection;

G3: Vehicle (saline); mice were sacrificed at 50 h post-infection;

G4: Vehicle (saline); mice were sacrificed at 74 h post-infection;

G5: Free Van HCl, 300 mg/kg of body weight; mice were sacrificed at 50 h post-infection;

G6: Free Van HCl, 300 mg/kg of body weight; mice were sacrificed at 74 h post-infection;

G7: Free Van HCl, 100 mg/kg of body weight; mice were sacrificed at 50 h post-infection;

G8: Free Van HCl, 100 mg/kg of body weight; mice were sacrificed at 74 h post-infection;

G9: Vancomycin pharmaceutical composition, comprising liposomal vancomycin 5A and second liposome suspension 5B at a lipid weight ratio of 1:1; diluted with saline to 20 mg/mL for dosing; 100 mg/kg of body weight; sacrificed at 50 h post-infection;

G10: Same vancomycin pharmaceutical composition as G9; diluted with saline to 20 mg/mL for dosing; 100 mg/kg of body weight; sacrificed at 74 h post-infection.

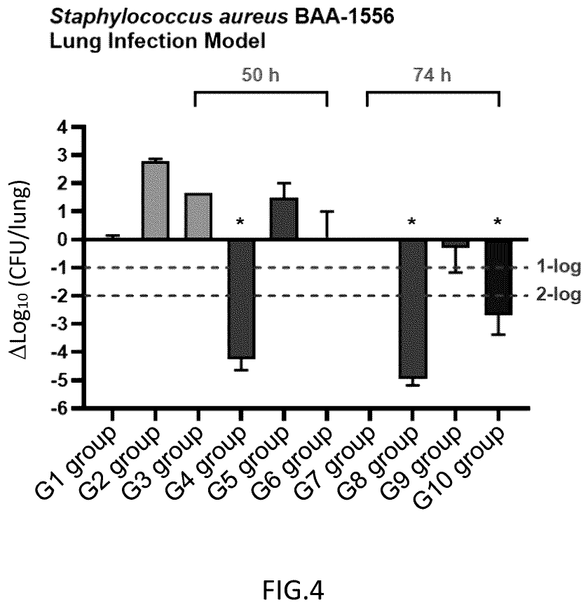

The logarithmic numbers of colony-forming units (CFU) per lung of G1 to G10 groups are calculated and the difference of the Log₁₀ treatment−Log₁₀ baseline (ΔLog₁₀ (CFU/lung)) is shown in FIG. 4. At 50 h and 74 h post-infection, the bacteria count data clearly indicated that the vancomycin pharmaceutical composition (100 mg/kg) is more effective than free Van HCl (100 mg/kg) in treating lung infections, and the vancomycin pharmaceutical composition indeed had a prolonged therapeutic effect. The viable bacteria counts compared to 2 h baseline group at 50 h were-4.25 log for free Van HCl 300 mg/kg, +1.49 log for free Van HCl 100 mg/kg, and +0.08 log for Liposomal Van 100 mg/kg, respectively. At 74 h, the viable bacteria counts compared to 2 h baseline group were-4.94 log (2 out of 5 mice even below the limit of detection) for free Van HCl 300 mg/kg, −0.29 log for free Van HCl 100 mg/kg, and −2.69 log for Liposomal Van 100 mg/kg, respectively.

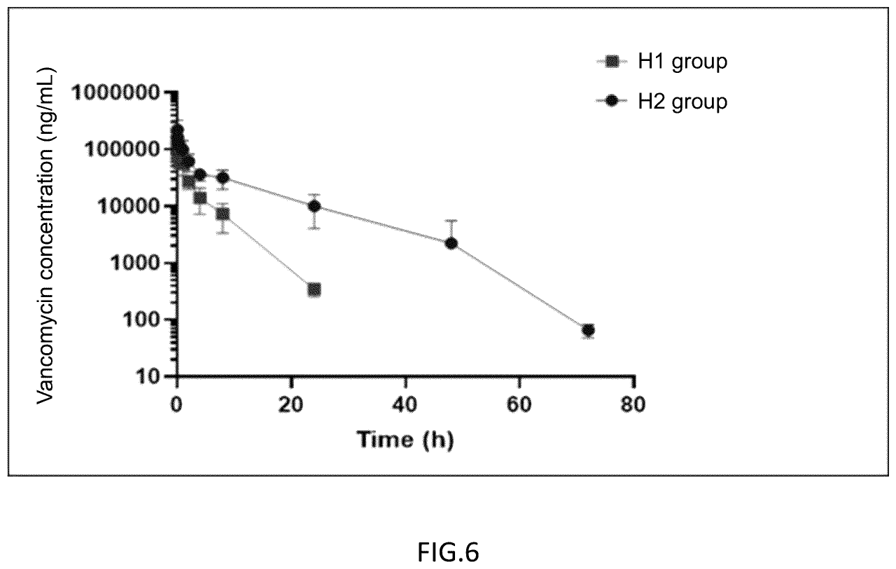

As shown in FIG. 6, the vancomycin lung tissue distribution data indicated there was about a 5× higher Van accumulation in lung tissues when comparing Liposomal Van 100 mg/kg to free Van HCl 100 mg/kg at both 50 h and 74 h. The accumulation level of free Van 300 mg/kg is about the same as Liposomal Van 100 mg/kg and appears declining. Results from this study clearly demonstrated the therapeutic potential of current invention to address the unmet medical need in lung infections (i.e., pneumonia) particularly those infected by MRSA, MISA or even VRSA.

### Animal Test 5: Pharmacokinetic Study of Reconstituted Liposomal Vancomycin in Beagle Dogs

Similar to the mice PK study in Animal Test 2, a comparative PK study in a (non-rodent) canine model per regulatory requirements was also performed.

In this test, 3 non-naïve beagle dogs (including 1 male: #1006 with body weight (BW) of 14.0 kg; and 2 females: #0007 BW14.2 kg and #0008 BW 9.7 kg) were administered with the following test compound or composition through 30-minute IV infusion. Blood samples were collected at specific time points during dosing (before infusion, in the middle of infusion and at the end of infusion) and after dosing, for examination of total vancomycin exposure levels in all plasma samples derived from the blood samples.

H1: free Van HCl, Van 33 mg/kg of body weight; and

H2: Vancomycin pharmaceutical composition, comprising liposomal vancomycin 6A and second liposome suspension 6B at a lipid weight ratio of 1:1; Van 33 mg/kg of body weight.

The plasma concentrations of H1 and H2 groups of this test are shown in FIG. 6; and the exposure levels of vancomycin in dog plasma samples of H1 and H2 groups were recorded in Tables 9 and 10, respectively. In this study, the levels of vancomycin are the levels of “total vancomycin”, including liposomal vancomycin (Lipo Van) and free form vancomycin (FreeVan).

The results of vancomycin exposure level over time are tabulated as Tables 10 and 11 below. The values of mean and standard deviation (SD) are calculated. BLOQ means “below the limit of quantification”, and N/A means “not available”.

TABLE 10
Vancomycin exposure level of plasma
samples of beagle dogs in H1 group

| | | Sample concentration (ng/mL) | | | | |
|---|---|---|---|---|---|---|
| | | Dog | Dog | Dog | | |
| Time (h) | | #1006 | #0007 | #0008 | Mean | SD |
| Before | 0 | BLOQ | BLOQ | BLOQ | BLOQ | N/A |
| infusion | | | | | | |
| Mid of | 0.25 | 128362 | 101476 | 99401 | 109746 | 16155 |
| infusion | | | | | | |
| End of | 0.5 | 138029 | 51901 | 212535 | 134155 | 80387 |
| infusion | | | | | | |
| After | 0.033 | 92966 | 82183 | 230045 | 135065 | 82432 |
| dosing | 0.083 | 130201 | 61593 | 228559 | 140118 | 83924 |
| | 0.25 | 57067 | 84222 | 89120 | 76803 | 17266 |
| | 0.5 | 52694 | 58057 | 62132 | 57628 | 4734 |
| | 1 | 40763 | 46506 | 79114 | 55461 | 20684 |
| | 2 | 22622 | 35677 | 23631 | 27310 | 7264 |
| | 4 | 10830 | 21949 | 9530 | 14103 | 6826 |
| | 8 | 4477 | 11770 | 5625 | 7291 | 3921 |
| | 24 | 262 | 447 | 333 | 347 | 93 |

TABLE 11
Vancomycin exposure level of plasma
samples of beagle dogs in H2 group

| | | Sample concentration (ng/mL) | | | | |
|---|---|---|---|---|---|---|
| | | Dog | Dog | Dog | | |
| Time (h) | | #1006 | #0007 | #0008 | Mean | SD |
| Before | 0 | BLOQ | BLOQ | 23 | 23 | N/A |
| infusion | | | | | | |
| Mid of | 0.25 | 103348 | 113860 | 143071 | 120093 | 20582 |
| infusion | | | | | | |
| End of | 0.5 | 154628 | 312264 | 253785 | 240226 | 79688 |
| infusion | | | | | | |
| After | 0.033 | 129553 | 131141 | 235193 | 165296 | 60538 |
| dosing | 0.083 | 169100 | 345568 | 154375 | 223014 | 106390 |
| | 0.25 | 105339 | 147703 | 126781 | 126608 | 21183 |
| | 0.5 | 121384 | 136749 | 78584 | 112239 | 30142 |
| | 1 | 70746 | 149993 | 75668 | 98802 | 44401 |
| | 2 | 85048 | 45482 | 53583 | 61371 | 20901 |
| | 4 | 26715 | 38869 | 43948 | 36511 | 8855 |
| | 8 | 19468 | 43425 | 32209 | 31701 | 11987 |
| | 24 | 3151 | 13550 | 13585 | 10095 | 6014 |
| | 48 | 116 | 536 | 6057 | 2236 | 3315 |
| | 72 | 56 | 56 | 86 | 66 | 17 |

In addition, the PK parameters of vancomycin in beagle dog plasma samples of H1 and H2 groups were recorded in Tables 12 and 13, respectively. The pharmacokinetics (PK) parameters listed in Tables 12 and 13 include t_{1/2}, C₀, AUClast, AUCInf, AUC/D, AUC Extr, MRT, Vss and CL. The definition of the PK parameter is the same as described above.

TABLE 12
PK parameters of H1 group

| | Sample concentration (ng/ml) | | | | |
|---|---|---|---|---|---|
| | Dog | Dog | Dog | | |
| | #1006 | #0007 | #0008 | Mean | SD |
| t_{1/2} (h) | 3.78 | 3.48 | 4.07 | 3.77 | 0.30 |
| C₀ (ng/mL) | 92966 | 99414 | 231031 | 141137 | 77917 |
| AUCₗₐₛₜ (h*ng/mL) | 195039 | 326583 | 262325 | 261316 | 65778 |
| AUC_{Inf} (h*ng/mL) | 196466 | 328826 | 264280 | 263191 | 66187 |
| AUC/D (h*kg*ng/mL/mg) | 5954 | 9964 | 8008 | 7975 | 2006 |
| AUC Extr (%) | 0.73 | 0.68 | 0.74 | 0.72 | 0.03 |
| MRT (h) | 3.53 | 4.60 | 3.16 | 3.76 | 0.75 |
| Vss (L/kg) | 0.59 | 0.46 | 0.39 | 0.48 | 0.10 |
| CL (mL/min/kg) | 2.80 | 1.67 | 2.08 | 2.18 | 0.57 |

TABLE 13
PK parameters of H2 group

| | Sample concentration (ng/mL) | | | | | Ratio of |
|---|---|---|---|---|---|---|
| | Dog | Dog | Dog | | | LipoVan/ |
| | #1006 | #0007 | #0008 | Mean | SD | FreeVan |
| t_{1/2} | 6.20 | 6.42 | 8.14 | 6.92 | 1.06 | 1.83 |
| (h) | | | | | | |
| C₀ | 129553 | 131141 | 310530 | 190408 | 104032 | 1.35 |
| (ng/mL) | | | | | | |
| AUCₗₐₛₜ | 615276 | 1143288 | 1096696 | 951753 | 292328 | 3.64 |
| (h*ng/mL) | | | | | | |
| AUC_{Inf} | 615777 | 1143807 | 1097707 | 952430 | 292460 | 3.62 |
| (h*ng/mL) | | | | | | |
| AUC/D | 18660 | 34661 | 33264 | 28862 | 8862 | 3.62 |
| (h*kg*ng/mL/mg) | | | | | | |
| AUC Extr | 0.08 | 0.05 | 0.09 | 0.07 | 0.02 | 0.10 |
| (%) | | | | | | |
| MRT | 6.42 | 10.00 | 15.49 | 10.64 | 4.57 | 2.83 |
| (h) | | | | | | |
| Vss | 0.34 | 0.29 | 0.47 | 0.37 | 0.09 | 0.76 |
| (L/kg) | | | | | | |
| CL | 0.89 | 0.48 | 0.50 | 0.63 | 0.23 | 0.29 |
| (mL/min/kg) | | | | | | |

It appears that the vancomycin pharmaceutical composition of the H2 group demonstrated improved PK parameters. In comparison with the H1 group, the t_{1/2} increased from 3.77 h to 6.92 h; and Co and AUCinf increased to 1.35× and 3.62×, respectively. A significantly prolonged circulation attributed to vancomycin pharmaceutical composition was also clearly demonstrated (also see FIG. 6). The enhanced PK is similar and could be more profound than in mice.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A vancomycin pharmaceutical composition, comprising a liposomal vancomycin, wherein the liposomal vancomycin comprises a therapeutically effective amount of vancomycin or a pharmaceutical acceptable salt thereof, and a first liposome comprising sphingomyelin and cholesterol.

2. The vancomycin pharmaceutical composition as claimed in claim 1, wherein, the weight ratio of the vancomycin to the first liposome is from about 9:1 to about 0.06:1.

3. The vancomycin pharmaceutical composition as claimed in claim 1, wherein the first liposome comprises 40 wt % to 60 wt % of cholesterol and 60 wt % to 40 wt % of sphingomyelin.

4. The vancomycin pharmaceutical composition as claimed in claim 1, wherein the liposomal vancomycin has a Z-average size of 60 nm to 180 nm.

5. The vancomycin pharmaceutical composition as claimed in claim 1, further comprising a second liposome, wherein the second liposome comprises sphingomyelin.

6. The vancomycin pharmaceutical composition as claimed in claim 5, wherein the second liposome further comprises a lipid selected from cholesterol, phospholipids, phospholipid derivatives, or a combination thereof.

7. The vancomycin pharmaceutical composition as claimed in claim 1, wherein the vancomycin pharmaceutical composition has an encapsulating efficiency of 3% to 25%.

8. The vancomycin pharmaceutical composition as claimed in claim 1, wherein the vancomycin pharmaceutical composition has a pH value of 3.0 to 7.0.

9. The vancomycin pharmaceutical composition as claimed in claim 1, which is administered through injection or inhalation.

10. A kit of a vancomycin pharmaceutical composition, comprising:
(a) a liposomal vancomycin, comprising a therapeutically effective amount of vancomycin or a pharmaceutical acceptable salt thereof, and a first liposome comprising sphingomyelin and cholesterol; and
(b) a second liposome, comprising sphingomyelin;
wherein (a) the liposomal vancomycin and (b) the second liposome are separately stored.

11. The kit as claimed in claim 10, wherein the first liposome further comprises a pharmaceutically acceptable excipient.

12. The kit as claimed in claim 10, wherein, in the liposomal vancomycin, the weight ratio of the vancomycin to the first liposome is from about 9:1 to about 0.06:1.

13. The kit as claimed in claim 10, wherein the first liposome comprises 40 wt % to 60 wt % of cholesterol and 60 wt % to 40 wt % of sphingomyelin.

14. The kit as claimed in claim 10, wherein the liposomal vancomycin in a liquid suspension form has a Z-average size from 60 nm to 180 nm.

15. The kit as claimed in claim 10, wherein the liposomal vancomycin has an encapsulating efficiency of 3% to 25% after reconstitution with the second liposome in a liquid suspension form or a reconstitution liquid selected from water and an intravenous fluid.

16. The kit as claimed in claim 10, wherein the second liposome further comprises a lipid selected from cholesterol, phospholipids, phospholipid derivatives, or a combination thereof.

17. The kit as claimed in claim 10, wherein the liposomal vancomycin is in a lyophilized solid form, and the second liposome is in a lyophilized solid form or a liquid suspension form.

18. The kit as claimed in claim 10, further comprising one or more portions of (c) a reconstitution liquid selected from water or an intravenous fluid.

19. The kit as claimed in claim 10, wherein the liposomal vancomycin in a lyophilized solid form is reconstituted with the second liposome in a liquid suspension form or a reconstitution liquid selected from water and an intravenous fluid, to obtain a liposomal vancomycin in a liquid suspension form; when the second liposome in a liquid suspension form is used to reconstitute the liposomal vancomycin in a lyophilized solid form, the second liposome in the kit is in a liquid suspension form.

20. A method for treating bacterial infection, comprising: administering the vancomycin pharmaceutical composition as claimed in claim 1 to a subject in need thereof.

21. A method for treating bacterial infection, comprising: providing the kit as claimed in claim 10; reconstituting the liposomal vancomycin with the second liposome to obtain a vancomycin pharmaceutical composition; and administering the vancomycin pharmaceutical composition to a subject in need thereof.

22. A method for treating bacterial infection, comprising: providing the kit as claimed in claim 10; reconstituting the liposomal vancomycin with a reconstitution liquid selected from water and an intravenous fluid to obtain a reconstituted liposomal vancomycin; and administering the reconstituted liposomal vancomycin and the second liposome to a subject in need thereof separately or simultaneously.
